(19) United States
(12) Patent Application Publication
(10) Pub. No.: US 2025/0003985 A1
(43) Pub. Date: Jan. 2, 2025

(54) Assays for Quantification of Anti-HPA-1a Antibodies

(71) Applicant: Rallybio IPA, LLC, New Haven, CT (US)

(72) Inventors: Douglas L. Sheridan, Branford, CT (US); Jonathan Reeves, Hawkesbury (CA); Julien Rainville Sirois, Laval (CA)

(21) Appl. No.: 18/707,589

(22) PCT Filed: Nov. 7, 2022
(86) PCT No.: PCT/US2022/079374

§ 371 Date: May 5, 2024

Related U.S. Application Data
(60) Provisional application No. 63/276,861, filed on Nov. 8, 2021.

(51) Int. Cl. G01N 33/68; C12Q 1/6804; G01N 33/543; G01N 33/58

(52) U.S. Cl. CPC G01N 33/6857; C12Q 1/6804; G01N 33/543; G01N 33/581; G01N 2333/70557; G01N 2333/908

(57) ABSTRACT

Methods of quantifying anti-HPA-1a antibodies in a sample, comprising (a) contacting the sample with a capture reagent that binds to the anti-HPA-1a antibodies to form a detection complex, wherein the capture reagent comprises a modified integrin psi and (b) measuring an amount of the detection complex, wherein the amount of the detection complex indicates the quantity of the anti-HPA-1a antibodies in the sample. The methods may also comprise contacting the sample with a detecting reagent that binds to the anti-HPA-1a antibodies and becomes part of the detection complex. The capture reagent and/or the detecting reagent may comprise a detection label, in which measuring the amount of the detection complex comprises measuring the amount of the detectable label.

### CROSS-REFERENCE TO RELATED APPLICATIONS

This application claims the benefit of priority of U.S. Provisional Patent Application No. 63/276,861 filed on 8 Nov. 2021, the content of which is hereby incorporated by reference in its entirety.

### SEQUENCE LISTING

The instant application contains a Sequence Listing, which has been submitted electronically in XML format and is hereby incorporated by reference in its entirety. Said XML copy, created on 10 Oct. 2022, is named IPA_006_WO1-Sequence_Listing, and is 17,679 bytes in size.

### BACKGROUND

Glycoprotein IIb/IIIa (GPIIb/IIIa), also known as integrin αIIbβ3, is a transmembrane platelet membrane glycoprotein that binds fibrinogen and von Willebrand factor, and plays a role in platelet activation. Human platelet antigen-1 (HPA-1) is defined by a single nucleotide polymorphism, resulting in a leucine to proline substitution at position 33 of integrin β₃ (GPIIIa). (Newman et al., 1989). Carriers of a leucine at position 33 of GPIIIa are defined as HPA-1a positive (HPA-1aa or HPA-1ab), whereas homozygous carriers of a proline at position 33 of GPIIIa are defined as HPA-1a negative (HPA-1 bb).

Mismatch between fetal and maternal HPA-1 alloantigens, where an HPA-1a-negative woman carries a fetus having a paternally-inherited HPA-1a allotype, can lead to maternal production of anti-HPA-1a antibodies. These antibodies can traverse the placenta, bind fetal platelets, and accelerate platelet destruction, causing fetal neonatal alloimmune thrombocytopenia (FNAIT). Maternal production of anti-HPA-1a antibodies is the most common cause of FNAIT, accounting for 85%-95% of severe FNAIT cases (Ghevaert et al., 2007).

There is currently no prophylactic treatment for FNAIT; however, administration of polyclonal or monoclonal anti-HPA-1a antibodies to an HPA-1a negative mother has been contemplated (U.S. Pat. Nos. 9,834,613; 10,882,919). Development of an anti-HPA-1a antibody-based therapeutic product will require assays with high levels of sensitivity and specificity in order to assess the potency of a potential therapeutic antibody product, to measure pharmacokinetic parameters in subjects to whom the antibody product has been administered, and to determine if the treatment has been effective in preventing alloimmunization.

The standard method for quantifying anti-platelet antibody concentrations is the monoclonal antibody-specific immobilization of platelet antigens (MAIPA) assay (Kiefel et al., 1987; Campbell et al., 2007). The MAIPA assay involves using monoclonal antibodies to attach to the same platelet antigen—but at a different binding site—as the anti-platelet antibody of interest, and to immobilize the bound platelet antigens on a substrate; the platelet antigen can then be detected using an enzyme-labeled anti-human immunoglobulin that binds to the anti-platelet antibody of interest (Kiefel et al., 1987). However, reproducibility and sensitivity with the MAIPA assay are known to be somewhat problematic (Campbell et al., 2007). In addition, detection and quantification of anti-HPA antibodies is complicated by the fact that these epitopes are present on membrane-bound proteins that are obligate heterodimers, and not easily isolated or manipulated in vitro. Therefore, the MAIPA assay must utilize human platelets as the source of HPA antigens, the disadvantages of which include instability of platelets in vitro, the requirement for human donors, and the inherent variability arising from using primary human tissues as a source of antigen.

The unavailability of a stable, soluble form of HPA-1a for use as a capture antigen in immunoassays has hampered the development of an accurate, precise, and validated method for quantifying and determining potency of anti-HPA-1a antibodies, as required to assess the pharmacokinetics and efficacy of potential therapeutics for bleeding disorders, such as FNAIT. Such a method has been absent from the field.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The invention provides accurate, precise, and highly selective assays for quantification of anti-HPA-1a antibodies, or for determination of the potency of anti-HPA-1a antibodies.

In one aspect, the invention provides a method of quantifying anti-HPA-1a antibodies in a sample. The method comprises (a) contacting the sample with a capture reagent that binds to the anti-HPA-1a antibodies to form a detection complex, in which the capture reagent comprises a modified integrin β3; and (b) measuring an amount of the detection complex. The amount of the detection complex indicates the quantity of the anti-HPA-1a antibodies in the sample.

In another aspect, the invention provides a method of determining potency of anti-HPA-1a antibodies in a sample. The method comprises (a) contacting the sample with a capture reagent that binds to the anti-HPA-1a antibodies to form a detection complex, in which the capture reagent comprises a modified integrin β; and (b) measuring an amount of the detection complex. The amount of the detection complex indicates the potency of the anti-HPA-1a antibodies in the sample.

In yet another aspect, the invention provides a method of measuring pharmacokinetics of anti-HPA-1a antibodies administered to a subject. The method comprises (a) contacting a sample from the subject with a capture reagent that binds to the anti-HPA-1a antibodies to form a detection complex, in which the capture reagent comprises a modified integrin β3; and (b) measuring an amount of the detection complex. The amount of the detection complex indicates the quantity of the anti-HPA-1a antibodies in the sample, and the quantity of the anti-HPA-1a antibodies in the sample is used to calculate the pharmacokinetics of the anti-HPA-1a antibodies administered to the subject.

The modified integrin β₃ may be prepared by replacing the I-like domain of integrin β₃ (βI) with the I domain of an integrin α (αI). In some embodiments, the modified integrin β₃ comprises the amino acid sequence of SEQ ID NO: 2. In some embodiments, the modified integrin β₃ consists of the amino acid sequence of SEQ ID NO: 2.

In some embodiments, the modified integrin β₃ is deglycosylated.

The capture reagent may comprise a detectable label attached to the modified integrin β₃, and measuring the amount of the detection complex comprises measuring the amount of the detectable label.

In some embodiments, the method further comprise contacting the sample with a detecting reagent that binds to the anti-HPA-1a antibodies to become part of the detection complex. The detecting reagent may comprise an antibody or antigen-binding fragment thereof that binds to the anti-HPA-1a antibodies. In certain embodiments, the detecting reagent comprises a detectable label attached to the antibody or antigen-binding fragment thereof, and measuring the amount of the detection complex comprises measuring the amount of the detectable label.

In some embodiments, the detectable label is selected from a group consisting of an enzyme, a fluorescent label, a chemiluminescent label, a bioluminescent label, a radioactive label, or a combination thereof. In certain embodiments, the detectable label comprises horseradish peroxidase.

In some embodiments, the amount of the detectable label is measured using light scattering, optical absorbance, fluorescence, chemiluminescence, electrochemiluminescence, or radioactivity.

The capture reagent may be immobilized on a solid support. In some embodiments, the solid support comprises a surface, particles, or a porous matrix. In certain embodiments, the solid support comprises a multi-well plate.

The method may further comprise attaching a first fragment of a reporter protein to the anti-HPA-1a antibodies. In some embodiments, the capture reagent comprises a second fragment of the reporter protein attached to the modified integrin β₃, and measuring the amount of the detection complex comprises measuring the amount of the reporter proteins that are formed when the capturing reagents bind to the anti-HPA-1a antibodies. In certain embodiments, the method additionally comprise contacting the sample with a detecting reagent that binds to the anti-HPA-1a antibodies to become part of the detection complex. The detecting reagent may comprise an antibody or antigen-binding fragment thereof that binds to the anti-HPA-1a antibodies, and a second fragment of the reporter protein attached to the antibody or antigen-binding fragment thereof, such that measuring the amount of the detection complex comprises measuring the amount of the reporter proteins that are formed when the detecting reagents bind to the anti-HPA-1a antibodies. The reporter protein may be selected from beta-lactamase, dihydrofolate reductase, focal adhesion kinase, Gal4, green fluorescent protein, horseradish peroxidase, infrared fluorescent protein IFP1.4, lacZ (beta-galactosidase), luciferase, tobacco etch virus protease, and ubiquitin. The amount of the reporter protein may be measured using light scattering, optical absorbance, fluorescence, chemiluminescence, electrochemiluminescence, or radioactivity.

In some embodiments, the capture reagent comprises an oligonucleotide attached to the modified integrin β₃, and measuring the amount of the detection complex comprises measuring the amount of the oligonucleotide. The method may further comprise contacting the sample with a detecting reagent that binds to the anti-HPA-1a antibodies to become part of the detection complex. In certain embodiments, the detecting reagent comprises an antibody or antigen-binding fragment thereof that binds to the anti-HPA-1a antibodies, and an oligonucleotide attached to the antibody or antigen-binding fragment thereof, such that measuring the amount of the detection complex comprises measuring the amount of the oligonucleotide. In particular embodiments, the amount of the oligonucleotide is measured using real-time polymerase chain reaction (PCR).

In some embodiments, the antibody or antigen-binding fragment thereof comprises an anti-immunoglobulin G (IgG) antibody, anti-IgM antibody, anti-IgD antibody, anti-IgE antibody, or anti-IgA antibody. In certain embodiments, the antibody or antigen-binding fragment thereof comprises an anti-IgG antibody.

The sample may comprise a biological fluid selected from whole blood, serum, and plasma.

In some embodiments, the anti-HPA-1a antibodies are polyclonal antibodies.

In some embodiments, the anti-HPA-1a antibodies are monoclonal antibodies. The monoclonal anti-HPA-1a antibodies may comprise (i) a variable heavy (VH) complementarity determining region (CDR) 1 that has the amino acid sequence of SEQ ID NO: 6, (ii) a VH CDR2 that has the amino acid sequence of SEQ ID NO: 7, (iii) a VH CDR3 that has the amino acid sequence of SEQ ID NO: 8, (iv) a variable light (VL) CDR1 that has the amino acid sequence of SEQ ID NO: 9, (v) a VL CDR2 that has the amino acid sequence of SEQ ID NO: 10, and (vi) a VL CDR3 that has the amino acid sequence of SEQ ID NO: 11 in which the CDRs are defined by IMGT. In certain embodiments, the monoclonal anti-HPA-1a antibodies may comprise a heavy chain variable region comprising SEQ ID NO: 4, or a sequence having at least 80% sequence identity thereto; and a light chain variable region comprising SEQ ID NO: 5, or a sequence having at least 80% sequence identity thereto.

In certain embodiments, the monoclonal antibodies are mAb 26.4.

In embodiments of the invention, the method of quantifying anti-HPA-1a antibodies in a sample comprises (a) contacting the sample with a capture reagent that binds to the anti-HPA-1a antibodies, in which the capture reagent comprises a modified integrin β₃ comprising the amino acid sequence of SEQ ID NO: 2; (b) contacting the sample with a detecting reagent that binds to the anti-HPA-1a antibodies, in which the detecting reagent comprises an antibody or antigen-binding fragment thereof that binds to the anti-HPA-1a antibodies and a detectable label attached thereto, and wherein the anti-HPA-1a antibodies, the capture reagent, and the detecting reagent forms a detection complex; and (c) measuring an amount of the detection complex, such that the amount of the detection complex indicates the quantity of the anti-HPA-1a antibodies in the sample.

In embodiments of the invention, the method of determining potency of anti-HPA-1a antibodies in a sample comprises (a) contacting the sample with a capture reagent that binds to the anti-HPA-1a antibodies, in which the capture reagent comprises a modified integrin β₃ comprising the amino acid sequence of SEQ ID NO: 2; (b) contacting the sample with a detecting reagent that binds to the anti-HPA-1a antibodies, in which the detecting reagent comprises an antibody or antigen-binding fragment thereof that binds to the anti-HPA-1a antibodies and a detectable label attached thereto, and the anti-HPA-1a antibodies, the capture reagent, and the detecting reagent forms a detection complex; and (c) measuring an amount of the detection complex, such that the amount of the detection complex indicates the potency of the anti-HPA-1a antibodies in the sample.

In embodiments of the invention, the method of measuring pharmacokinetics of anti-HPA-1a antibodies administered to a subject comprises (a) contacting a sample from the subject with a capture reagent that binds to the anti-HPA-1a antibodies, in which the capture reagent comprises a modified integrin β₃ comprising the amino acid sequence of SEQ ID NO: 2; (b) contacting the sample with a detecting reagent that binds to the anti-HPA-1a antibodies, in which the detecting reagent comprises an antibody or antigen-binding fragment thereof that binds to the anti-HPA-1a antibodies and a detectable label attached thereto, and the anti-HPA-1a antibodies, the capture reagent, and the detecting reagent forms a detection complex; and (c) measuring an amount of the detection complex, such that the amount of the detection complex indicates the quantity of the anti-HPA-1a antibodies in the sample.

In a further aspect, the invention provides a kit for an assay for quantifying anti-HPA-1a antibodies in a sample, the kit comprising: (i) a capture reagent comprising a modified integrin β3; and (ii) instructions on how to perform the assay using the capture reagent.

The modified integrin β₃ may be prepared by replacing the I-like domain of integrin β₃ (βI) with the I domain of an integrin α (αI). In certain embodiments, the modified integrin β₃ comprises the amino acid sequence of SEQ ID NO: 2. In particular embodiments, the modified integrin β₃ consists of the amino acid sequence of SEQ ID NO: 2.

In some embodiments, the capture reagent further comprises a detectable label attached to the modified integrin cβ₃.

In some embodiments, the kit further comprises a detecting reagent comprising an antibody or antigen-binding fragment thereof that binds to anti-HPA-1a antibodies. In certain embodiments, the detecting reagent further comprises a detectable label attached to the antibody or antigen-binding fragment thereof.

The detectable label may comprise an enzyme. In some embodiments the kit may further comprise a substrate required by the enzyme to produce a detectable signal.

In some embodiments, the capture reagent is immobilized on a solid support. In other embodiments, the kit comprises a solid support on which to immobilize the capture reagent.

The kit may also comprise washing buffers, incubation buffers, blocking agents, or a combination thereof.

In yet another aspect, the invention provides a method of manufacturing a pharmaceutical composition comprising an anti-HPA-1a antibody. The method comprises (a) admixing an anti-HPA-1a antibody and a pharmaceutically acceptable carrier to prepare a composition; (b) measuring the potency anti-HPA-1a antibody in the composition according to the methods of the invention to determine the concentration of anti-HPA-1a antibody in the composition; and (c) optionally adjusting the concentration of anti-HPA-1a antibody in the composition to a specification concentration for a pharmaceutical composition comprising anti-HPA-1a antibody.

In some embodiments, the pharmaceutical composition comprises a monoclonal anti-HPA-1a antibody. The monoclonal anti-HPA-1a antibody may comprise (i) a variable heavy (VH) complementarity determining region (CDR) 1 that has the amino acid sequence of SEQ ID NO: 6, (ii) a VH CDR2 that has the amino acid sequence of SEQ ID NO: 7, (iii) a VH CDR3 that has the amino acid sequence of SEQ ID NO: 8, (iv) a variable light (VL) CDR1 that has the amino acid sequence of SEQ ID NO: 9, (v) a VL CDR2 that has the amino acid sequence of SEQ ID NO: 10, and (vi) a VL CDR3 that has the amino acid sequence of SEQ ID NO: 11 in which the CDRs are defined by IMGT. In certain embodiments, the monoclonal anti-HPA-1a antibody may comprise a heavy chain variable region comprising SEQ ID NO: 4, or a sequence having at least 80% sequence identity thereto; and a light chain variable region comprising SEQ ID NO. 5, or a sequence having at least 80% sequence identity thereto.

In some embodiments, the pharmaceutical composition comprises a polyclonal anti-HPA-1a antibody. The polyclonal anti-HPA-1a antibody may be from plasma of one or more HPA-1a-negative subjects immunized with HPA-1a. In certain embodiments, the subject was alloimmunized with HPA-1a, or immunized with HPA-1a positive platelets, or immunized with a purified or recombinant preparation HPA-1a antigen. In certain embodiments, the polyclonal anti-HPA-1a antibody was cleared of viruses.

### BRIEF DESCRIPTION OF THE DRAWINGS

FIG. 1 shows a representative calibration curve generated from an ELISA assay for measuring anti-HPA-1a gamma globulins in a matrix comprising blocker casein in phosphate buffer solution (“casein-PBS”) in accordance with embodiments of the present invention, as described in Example 2.

FIG. 2 shows a representative calibration curve generated from an ELISA assay for measuring mAb 26.4 in a matrix comprising casein-PBS in accordance with embodiments of the present invention, as described in Example 2.

FIG. 3 shows a representative calibration curve generated from an ELISA assay for measuring the WHO Standard in a matrix comprising casein-PBS in accordance with embodiments of the present invention, as described in Example 2.

FIG. 4 shows a representative calibration curve generated from an ELISA assay for measuring anti-HPA-1a gamma globulins in a matrix comprising mouse serum in accordance with embodiments of the present invention, as described in Example 3.

FIG. 5 shows a representative calibration curve generated from an ELISA assay for measuring mAb 26.4 in a matrix comprising mouse serum in accordance with embodiments of the present invention, as described in Example 4.

### DETAILED DESCRIPTION OF THE INVENTION

The practice of the present invention will employ, unless otherwise indicated, conventional techniques of pharmaceutics, formulation science, protein chemistry, cell biology, cell culture, molecular biology, microbiology, recombinant DNA, and immunology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### I. Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A. B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The terms “polypeptide,” “peptide,” and “protein” are used interchangeably herein to refer to polymers of amino acids of any length. The polymer can be linear or branched, can comprise modified amino acids, and can be interrupted by non-amino acids. Except where indicated otherwise, e.g., for the abbreviations for the uncommon or unnatural amino acids set forth herein, the three-letter and one-letter abbreviations, as used in the art, are used herein to represent amino acid residues. Groups or strings of amino acid abbreviations are used to represent peptides. Except where specifically indicated, peptides are indicated with the N-terminus of the left and the sequence is written from the N-terminus to the C-terminus.

A “polynucleotide,” as used herein can include one or more “nucleic acids,” “nucleic acid molecules,” or “nucleic acid sequences,” and refers to a polymer of nucleotides of any length, and includes DNA and RNA The polynucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin” are used interchangeably herein.

A typical antibody is composed of two identical pairs of polypeptide chains, each pair having one “heavy” chain and one “light” chain. Each chain is comprised of a variable region, which forms the antibody binding site, and a constant region, which can mediate the binding of the antibody to host tissues or factors. Immunoglobulin molecules can be divided into classes depending on the constant region of the heavy chain. The classes are immunoglobulin gamma (IgG), immunoglobulin mu (IgM), immunoglobulin delta (IgD), immunoglobulin epsilon (IgE), and immunoglobulin alpha (IgA). The heavy chain constant regions differ structurally and antigenically among the subclasses. IgG is the main type of antibody found in blood and extracellular fluid, and it plays a central role in the humoral immune response.

As used herein, the term “antibody” encompasses polyclonal antibodies; monoclonal antibodies; multi-specific antibodies, such as bispecific antibodies generated from at least two intact antibodies, chimeric antibodies, fusion proteins comprising an antigen-determination portion of an antibody; and any other modified immunoglobulin molecule comprising a particular paratope.

A “monoclonal antibody” (mAb) refers to a homogeneous antibody population that is involved in the highly specific recognition and binding of a single antigenic determinant (epitope). “Polyclonal antibodies” are a mixture of monoclonal antibodies directed against different epitopes of the same antigen. The term “monoclonal” can apply to full-length monoclonal antibodies, as well as to antigen-binding fragments, fusion proteins comprising an antigen-binding region, and any other modified immunoglobulin molecule comprising an antigen recognition site.

The term “antigen-binding fragment” refers to a portion of an intact antibody comprising the complementarity determining regions (CDRs) of the antibody. Examples of antigen-binding fragments include Fab, Fab′, F(ab′)2, and Fv fragments, linear antibodies, single chain antibodies (e.g., ScFvs), and multi-specific antibodies formed from antibody fragments. The antigen-binding fragments are defined using various terms and numbering schemes as follows:
(i) Kabat: CDRs are based on sequence variability (Wu and Kabat, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” or “HV” refers to the regions of an antibody variable domain which are hypervariable in structure as defined by Chothia and Lesk (Chothia and Lesk, 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin, 2008);
(iii) IMGT: Another definition of the regions that form the antigen-binding site has been proposed by Lefranc (Lefranc et al., 2003) based on the comparison of V domains from immunoglobulins and T-cell receptors. The Intemational ImMunoGeneTics (IMGT) database provides a standardized numbering and definition of these regions. The correspondence between CDRs, HVs and IMGT delineations is described in Lefranc et al., 2003, Id.;
(iv) AbM: A compromise between Kabat and Chothia numbering schemes is the AbM numbering convention described by Martin (Martin, 2010).
(v) The antigen-binding site can also be delineated based on “Specificity Determining Residue Usage” (SDRU) (Almagro, 2004), where SDR, refers to amino acid residues of an immunoglobulin that are directly involved in antigen contact.

“Binding affinity” generally refers to the strength of the sum total of non-covalent interactions between a single binding site of a molecule and its binding partner (e.g., a receptor and its ligand, an antibody and its antigen, two monomers that form a dimer, etc.). Unless indicated otherwise, as used herein, “binding affinity” refers to intrinsic binding affinity which reflects a 1:1 interaction between members of a binding pair. The affinity of a molecule X for its partner Y can generally be represented by the dissociation constant (K_{D}). Affinity can be measured by common methods known in the art, including those described herein. Low-affinity binding partners generally bind slowly and tend to dissociate readily, whereas high-affinity binding partners generally bind faster and tend to remain bound longer.

The affinity or avidity of a molecule for its binding partner can be determined experimentally using any suitable method known in the art, e.g., flow cytometry, enzyme-linked immunosorbent assay (ELISA), or radioimmunoassay (RIA), or kinetics (e.g., KINEXA® or BIACORE™ or OCTET® analysis). Direct binding assays as well as competitive binding assay formats can be readily employed. (See, e.g., Berzofsky et al., “Antibody-Antigen Interactions,” In Fundamental Immunology, Paul, W. E., ed., Raven Press: New York, N.Y. (1984); Kuby. Immunology, W. H. Freeman and Company: New York, N.Y. (1992)). The measured affinity of a particular binding pair interaction can vary if measured under different conditions (e.g., salt concentration, pH, temperature). Thus, measurements of affinity and other binding parameters (e.g., K_{D} or Kd, Kₒₙ, K_{off}) are made with standardized solutions of binding partners and a standardized buffer, as known in the art.

The terms “inhibit,” “block,” and “suppress” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

As used herein, “quantify” or “quantity” refers to the measurement of the amount of antibody in a sample, and is typically calculated as a concentration such as a mass concentration (mass of the antibody divided by volume of the sample). Generally, quantity is determined in a sample that is collected from a subject.

As used herein, “potency” is used synonymously with “concentration” and indicates the ability of antibody to bind to its cognate antigen. Generally, potency is determined in a sample that is manufactured, such as in pharmaceutical compositions. Potency is typically measured in international units. Potency also means “relative potency.”

As used herein, “specification concentration” refers to a concentration or range of concentrations to which a substance or product, such as a pharmaceutical composition comprising anti-HPA-1a antibody, should conform to be considered acceptable for its intended use. Generally, the specification concentration for the substance or product is approved by a regulatory authority, such as the Food and Drug Administration.

The term “international unit” or “IU” is a unit of measurement of the amount of a substance as determined by its activity. The mass or volume that constitutes one international unit of a substance will vary based on the substance that is being measured. For anti-HPA-1a antibodies, the amount of antibodies in one IU is set to an international standard (Allen et al. 2005) adopted by the World Health Organization (see WHO International Standard: Anti-HPA-1a Standard (100 IU)).

As used herein, “WHO Standard” refers to the WHO International Standard of anti-HPA-1a antibodies, prepared by pooling human plasma collected from six donors immunized against HPA-1a (see WHO International Standard: Anti-HPA-1a Standard (100 IU)).

An “isolated” molecule is one that is in a form not found in nature, including those which have been purified.

A “label” is a detectable compound that can be conjugated directly or indirectly to a molecule, so as to generate a “labeled” molecule. The label can be detectable on its own (e.g., radioisotope labels or fluorescent labels), or can be indirectly detected, for example, by catalyzing chemical alteration of a substrate compound or composition that is detectable (e.g., an enzymatic label) or by other means of indirect detection (e.g., biotinylation).

An “active agent” is an ingredient that is intended to furnish biological activity. The active agent can be in association with one or more other ingredients.

An “effective amount” of an active agent is an amount sufficient to carry out a specifically stated purpose.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g. acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

The terms “alloimmune response” or “alloimmunization” is an immune response to non-self antigens that are from the same species. As a result, the body produces antibodies against the non-self antigens.

The human amino acid sequence of GPIIIa (integrin β₃) is set forth in GenBank accession no. AAA52589.1, which includes a 26-amino acid signal peptide. HPA-1 is a polymorphism at position 33 of the mature integrin β3 chain (SEQ ID NO: 1). Individuals who have a Leu at position 33 in one or more copies of ITGB3 (i.e., the gene that encodes integrin β3) or in any of their integrin β3 are “HPA-1a positive,” “positive for HPA-1a,” “HPA-1a,” or “HPA-1ab,” while individuals who do not have a Leu at position 33 (e.g., have a Pro at position 33) in all copies of ITGB3 or in all of their integrin β₃ are “HPA-1a negative” or “negative for HPA-1a.”

“Accuracy” as used herein refers to the degree of closeness of the determined value to the nominal or known true value. Accuracy can be assessed by determining the relative error (RE) of the measured concentration as compared to the theoretical concentration, using the following equation:

$$R E ( \% ) = \frac{( measured concentration ) - ( theoretical concentration )}{( theoretical concentration )} \times 100$$

“Precision” as used herein refers to the closeness of agreement among a series of measurements obtained from multiple sampling of the same sample. Precision can be assessed by determining the coefficient of variation (CV) of the measurements, using the following equation:

$$C V ( \% ) = \frac{standard deviation ( calculated between replicate values )}{( mean of replicate values )} \times 100$$

“Selectivity” as used herein refers to the extent to which the method can determine a particular compound in a sample without interference from other components in the sample.

“Sensitivity” as used herein refers to the lowest anti-HPA-1a antibody concentration in the sample that can be measured with acceptable accuracy and precision (i.e., lower limit of quantification, or LLOQ).

“Specificity” refers to the ability of the assay to assess the anti-HPA-1a antibodies in the presence of other components that are expected to be present (e.g., impurities, degradation products, sample components, etc.).

“Reproducibility” refers to the precision of the assay under the same operating conditions over a short period of time.

“Pharmacokinetics” refers to the study of how a substance, following administration to a body, enters the blood circulation (absorption), is dispersed or disseminated throughout the fluids and tissues of the body (distribution), how the substance is recognized and transformed by the body (metabolism), and is removed from the body (excretion). The substance may be a drug or, relevant to the present invention, anti-HPA-1a antibodies. Pharmacokinetics may be evaluated using various metrics, many of which are calculated based on the quantity of the substance in the body (e.g., in the plasma) at various time points following the administration of the substance.

“Cₘₐₓ” is a pharmacokinetic metric that refers to the peak plasma concentration of the substance after administration.

“Tₘₐₓ” is a pharmacokinetic metric that refers to the time after administration of the substance to reach Cₘₐₓ.

“AUC” or “area-under-the-curve” is a pharmacokinetic metric that describes the variation of the concentration of the substance in blood plasma as a function of time. AUC may be calculated for different periods of time, for example, from time zero to specified time t (AUCₜ), from time zero to infinity (AUC_{∞}), etc.

“Elimination half-life” or “half-life” or “t_{1/2}” is a pharmacokinetic metric that refers to the time required for the concentration of the substance to reach half of its original value.

“Clearance” is a pharmacokinetic metric that refers to the volume of plasma cleared of the substance per unit time. Clearance may be calculated by dividing the dose of the substance by AUC.

### II. Assay

The present invention relates to assays for measuring anti-HPA-1a antibodies in a sample, methods of using the assays, and kits thereof. The assays utilize a capture reagent that reliably binds to anti-HPA-1a antibodies. The invention surprisingly exhibits the accuracy, precision, and selectivity required for measuring the potency and pharmacokinetics of anti-HPA-1a antibodies.

### Integrin Structure

Integrins are obligate heterodimers, composed of an a subunit and a β subunit. In mammals, combinations of 18 different α subunits and 8 different β subunits form 24 different integrin α/β complexes (Takada et al., 2007). The N-terminal regions of the α and β subunits associate via interaction between a β-propeller domain of the a subunit and an I-like domain of the β subunit. Association between the β-propeller domain and the I-like domain is required for proper folding of the α and β subunits.

Certain integrin a subunits comprise an I domain, which domain is homologous to both the A domain of von Willebrand factor and the I-like domain of integrin β (Lee et al., 1995; Takagi et al., 2002; Takada et al., 2007). However, unlike the I-like domain, which cannot be expressed autonomously, the I domain can be expressed independently of other integrin domains (Takagi et al., 2002).

### Capture and Detecting Reagents

The assays of the present invention employ a monomeric capture reagent that binds specifically to anti-HPA-1a antibodies. The capture reagent comprises a “modified integrin β₃.” which is defined herein as an integrin β₃, preferably a human integrin β₃, that has been engineered to permit expression and folding of the integrin β₃ as a single-chain polypeptide. The amino acid sequence of the mature human integrin β₃ polypeptide is set forth in SEQ ID NO: 1.

In one embodiment, the modified integrin β₃ is engineered by replacing the I-like domain of integrin β₃ (βI) with the I domain of an integrin α (αI). For example, the αI domain can be from an integrin α selected from the group consisting of α1, α2, α10, α11, αL, αM, αX, αD, and αE. In a specific embodiment, the βI domain is replaced with the al domain of integrin αL. (Thinn et al., 2018; Zhou et al., 2018). In a certain embodiment, the modified integrin β₃ comprises the amino acid sequence set forth in SEQ ID NO: 2, referred to herein as “cβ₃.” In a particular embodiment, the modified integrin β₃ consists of the amino acid sequence set forth in SEQ ID NO: 2.

In one embodiment, the modified integrin β₃ is engineered by removing the specificity-determining loop (SDL) of the βI domain (Takagi et al., 2002). For example, the modified integrin β₃ can comprise an amino acid sequence in which residues 160-188 of SEQ ID NO: 1 are deleted.

Accordingly, some embodiments of the invention include a capture reagent comprising a modified integrin β₃, wherein the modified integrin β₃ is a chimeric integrin β₃, wherein the wild-type βI sequence has been replaced with an αI sequence. Some embodiments of the invention include a capture reagent comprising a modified integrin β₃, wherein the modified integrin β₃ lacks the SDL of the βI domain.

In some embodiments, the modified integrin β₃ is deglycosylated.

The modified integrin β₃ may be generated by methods known in the art, including by recombinant protein production using transient or stable cell lines. In addition to the modified integrin β₃, the capture reagent can comprise sequences to facilitate expression, detection, and/or purification.

For example, the capture reagent can comprise a signal peptide. The wild-type integrin β₃ signal peptide has the amino acid sequence:

(SEQ ID NO: 3)
MRARPRPRPLWVTVLALGALAGVGVG.

The capture reagent can comprise one or more affinity tags. For example, the affinity tag can be selected from a poly(His) tag, such as a Hisb tag, a maltose binding protein (MBP) tag, a chitin binding protein (CBP) tag, a glutathione-S-transferase (GST) tag, a Strep-tag®, a FLAG tag, and an epitope tag.

The capture reagent can comprise one or more protease cleavage sites. For example, the protease cleavage site can be selected from an enteropeptidase cleavage site, a thrombin cleavage site, a Factor Xa cleavage site, a tobacco etch virus (TEV) protease cleavage site, a human riinovirus 3C (HRV3C) protease cleavage site, a carboxypeptidase cleavage site and a DAPase cleavage site.

In embodiments of the invention, the capture reagent may be immobilized on a solid support, either directly immobilized or indirectly immobilized through secondary binding reagents, such as targeting reagents. For example, a capture reagent may be linked to or comprise a targeting reagent that binds to an immobilized targeting reagent complement on the support. The binding of a targeting reagent to its complement may be direct (for example, the targeting reagent may be streptavidin and the complement may be biotin) or indirect through a bridging agent (for instance, the targeting reagent and complement may be biotin, and the bridging reagent may be a multivalent biotin binding receptor such as streptavidin). In some embodiments, a targeting agent and its complement comprise a first oligonucleotide and a complementary oligonucleotide, a receptor-ligand pair, an antigen-antibody pair, a hapten-antibody pair, an epitope-antibody pair, a mimetope-antibody pair, an aptamer-target molecule pair, hybridization partners, or an intercalator-target molecule pair.

Various supports are suitable for use in the methods of the present invention. The support may be made from a variety of different materials including, but not limited to, polymers (e.g., polystyrene and polypropylene), ceramics, glass, and composite materials (e.g., carbon-polymer composites such as carbon-based inks), and may be in the form of surfaces, particles, porous matrices, etc. Suitable supports include objects such as an assay container (e.g., test tubes, cuvettes, flow cells. FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc. Other suitable supports include particles (including but not limited to colloids or beads) commonly used in other types of particle-based assays such as magnetic, polypropylene, and latex particles; materials typically used in solid-phase synthesis such as polystyrene and polyacrylamide particles; and materials typically used in chromatographic applications such as silica, alumina, polyacrylamide, polystyrene. The materials may also be a fiber such as a carbon fibril. In certain embodiments, the capture reagent is immobilized on a microtiter plate, and in particular a multi-well microtiter plate such as MICROTEST® or MAXISORP®.

In some embodiments of the invention, the capture reagent is not immobilized on a solid support. In such embodiments, the methods of the invention can be performed in solution.

In some embodiments, the capture reagent bound to an anti-HPA-1a antibody forms a detection complex. In some embodiments, the capture reagent may comprise a detectable label that is conjugated or otherwise attached to the modified integrin β₃.

In other embodiments, the detection complex further comprises a detecting reagent. The detecting reagent may comprise an antibody or antigen-binding fragment thereof that binds to anti-HPA-1a antibodies. The antibody may be an anti-IgG, anti-IgM, anti-IgD, anti-IgE, or anti-IgA antibody. In preferred embodiments, the antibody is an anti-IgG antibody.

In some embodiments, the detecting reagent may comprise a detectable label that is conjugated or otherwise attached to the antibody or antigen-binding fragment thereof that binds to anti-HPA-1a antibodies.

The detectable label may be used to measure the amount of the detection complex that is formed. The detectable label may be a luminescent label (e.g., fluorescent, phosphorescent, chemiluminescent, bioluminescent and electrochemiluminescent label), radioactive label, enzyme, particle, magnetic substance, electroactive species, or the like, including combinations thereof. Examples of detectable labels for use in the capture reagent and/or detecting reagent include, but are not limited to, radioisotopes ³²P, ¹⁴C, ¹²⁵I, ³H, and ¹³¹I; fluorophores such as rare-earth chelates or fluorescein and its derivatives, rhodamine and its derivatives, ruthenium, dansyl, and umbelliferone; luceriferases, e.g., firefly luciferase and bacterial luciferase; luciferin; 2,3-dihydrophthalazinediones; HRP; alkaline phosphatase; β-galactosidase; glucoamylase; lysozyme; saccharide oxidases, e.g., glucose oxidase, galactose oxidase, and glucose-6-phosphate dehydrogenase; heterocyclic oxidases such as uricase and xanthine oxidase, coupled with an enzyme that employs hydrogen peroxide to oxidize a dye precursor such as HRP, lactoperoxidase, or microperoxidase; biotin (detectable by, e.g., avidin, streptavidin, streptavidin-HRP, and streptavidin-β-galactosidase with MUG); spin labels; bacteriophage labels; stable free radical; and the like.

In some embodiments, the capture reagent or the detecting reagent may be attached (for example, covalently linked) to a fragment of a reporter protein, and the anti-HPA-1a antibodies may be attached (for example, covalently linked) to a different fragment of the reporter protein. Such use of a reporter protein with the capture reagent or detecting reagent may be for a protein-fragment complementation assay, in which the binding of the capture reagent to an anti-HPA-1a antibody, or the binding of the detecting reagent to an anti-HPA-1a antibody, brings the fragments of the reporter protein in close proximity to allow them to form a functional reporter protein whose activity can be measured. In such embodiments, the reporter protein forms part of the detection complex with the capture reagent and the anti-HPA-1a antibody, or forms part of the detection complex with the capture reagent, detecting reagent, and the anti-HPA-1a antibody. Examples of reporter proteins may include, but are not limited to, beta-lactamase, dihydrofolate reductase (DHFR), focal adhesion kinase (FAK), Gal4, green fluorescent protein (split-GFP), HRP, infrared fluorescent protein IFP1.4, lacZ (beta-galactosidase), luciferase (e.g., recombinase enhanced bimolecular luciferase, Gaussia princeps luciferase, etc.), tobacco etch virus protease (TEV), and ubiquitin.

In other embodiments, the capture reagent or the detecting reagent may be attached to an oligonucleotide. Such use of an oligonucleotide may be for an immune-PCR assay, in which the detection complex formed by the capture reagent and the anti-HPA-1a antibody, or formed by the capture reagent, detecting reagent, and the anti-HPA-1a antibody, is measured by amplifying and detecting the oligonucleotide via real-time PCR.

### Format

The assays of the present invention may involve a variety of devices and/or formats. The devices may include, e.g., assay modules, such as assay plates, cartridges, multi-well assay plates, reaction vessels, test tubes, cuvettes, flow cells, assay chips, lateral flow devices, etc., having assay reagents (which may include capture reagents or other binding reagents) added as the assay progresses or pre-loaded in the wells, chambers, or assay regions of the assay module. These devices may employ a variety of assay formats for specific binding assays, e.g., immunoassay or immunochromatographic assays, including ELISAs and electrochemiluminescence (ECL) based immunoassays.

The assays of the invention can be used with a variety of methods for measuring the amount of the detection complex, i.e., the amount of the anti-HPA-1a antibodies bound to the capture reagent, or the amount of the anti-HPA-1a antibodies bound to the capture reagent and to a detecting reagent. Techniques that may be used include, but are not limited to, techniques known in the art such as cell culture-based assays, binding assays (including agglutination tests, immunoassays, etc.), enzymatic assays, colorometric assays, etc.

Methods for measuring the amount of the detection complex include techniques that measure through the detection of detectable labels, i.e., labels in the capture reagent or the detecting reagent that is bound to the anti-HPA-1a antibodies. The labels may be detected by visualization (e.g., particles that may be seen visually and labels that generate a measurable signal such as light scattering, optical absorbance, fluorescence, chemiluminescence, electrochemiluminescence, radioactivity, magnetic fields, etc.), or by measurement of a signal produced by chemical activity such as light scattering, absorbance, fluorescence, etc.

Alternatively, methods for measuring the amount of the detection complex are label-free techniques, which include but are not limited to techniques that measure changes in mass or refractive index at a surface after binding of the anti-HPA-1a antibodies to an immobilized capture reagent (e.g., surface acoustic wave techniques, surface plasmon resonance sensors, ellipsometric techniques, etc.); mass spectrometric techniques (including techniques like matrix-assisted laser desorption/ionization (MALDI), surface-enhanced laser desorption/ionization (SELDI), etc., that can measure the anti-HPA-1a antibodies on a surface), chromatographic or electrophoretic techniques; etc.

In some embodiments, measurement of the amount of the detection complex comprises comparing the results of the detection method with a standard curve to determine the quantity of the anti-HPA-1a antibody compared to the known amount.

### Sandwich ELISA

In preferred embodiments, the amount of the detection complex is measured using the sandwich ELISA format. The capture reagent may be affixed to a support such as a plate, and the sample containing the anti-HPA-1a antibodies may be washed over the support so that it can bind to the capture reagent. A detecting reagent linked to an enzyme, such as HRP is then washed over the support so that it can bind to the anti-HPA-1a antibodies, and a substance is added that the enzyme converts to a product that provides a change in a detectable signal. As an example, if the enzyme is HRP, the substance may be TMB substrate. The formation of product may be detectable. e.g., due a difference, relative to the substrate, in a measurable property such as absorbance, fluorescence, chemiluminescence, light scattering, etc. Certain (but not all) measurement methods that may be used with support-binding methods according to the invention may benefit from or require a wash step to remove unbound components (e.g., labels) from the support.

The amount of the modified integrin β₃ in the capture reagents employed is sufficiently large to give a good signal in comparison with the standards, but not in molar excess compared to the maximum expected level of antibody of interest in the sample. In some embodiments, the capture reagent comprises modified integrin β₃ in an amount of about 0.01 μg/mL to about 10 μg/mL, or about 0.1 μg/mL to about 5 μg/mL, preferably about 0.5, 1, or 2 μg/mL.

The support may be treated with a blocking agent that binds non-specifically to and saturates the binding sites to prevent unwanted binding of the anti-HPA-1a antibodies to excess sites on the support. Examples of appropriate blocking agents for this purpose include, e.g., gelatin, bovine serum albumin, egg albumin, casein, and non-fat milk. The blocking treatment typically takes place under conditions of ambient temperatures for about one to five hours. In some embodiments, the blocking treatment includes use of an orbital shaker or the like at 50 to 200 RPM, preferably 100 RPM.

The conditions for incubation of the sample and the capture reagent are selected to maximize sensitivity of the assay and to minimize dissociation, and to ensure that any anti-HPA-1a antibodies in the sample binds to the capture reagent. Incubation is accomplished at generally constant temperatures, ranging from about 0° C. to about 40° C., preferably at or about room temperature. The time for incubation is generally no greater than about five hours, and is preferably about 0.5 to three hours, or about one hour.

Incubation of the detecting reagent with the capture reagent and bound anti-HPA-1a antibodies may occur at a temperature of about 20 to 40° C., preferably at or about room temperature. The time for incubation is generally no greater than about five hours, and is preferably about 0.5 to three hours, or about one hour.

### Methods of Use

Aspects of the present invention relate to the use of the assays to analyze anti-HPA-1a antibodies. One aspect of the invention relates to methods of quantifying anti-HPA-1a antibodies in a sample. The methods comprise (a) contacting the sample with a capture reagent that binds to anti-HPA-1a antibodies to form a detection complex, wherein the capture reagent comprises a modified integrin β₃; and (b) measuring an amount of the detection complex, wherein the amount of the detection complex indicates the quantity of the anti-HPA-1a antibodies in the sample.

Another aspect of the invention relates to methods of determining potency of anti-HPA-1a antibodies in a sample. The methods comprise (a) contacting the sample with a capture reagent that binds to anti-HPA-1a antibodies to form a detection complex, wherein the capture reagent comprises a modified integrin β₃; and (b) measuring an amount of the detection complex, wherein the amount of the detection complex indicates the potency of the anti-HPA-1a antibodies in the sample.

A further aspect of the invention relates to methods of measuring pharmacokinetics of anti-HPA-1a antibodies administered to a subject. The methods comprise (a) contacting a sample from the subject with a capture reagent that binds to anti-HPA-1a antibodies to form a detection complex, w % herein the capture reagent comprises a modified integrin 133, and (b) measuring an amount of the detection complex, wherein the amount of the detection complex indicates the quantity of the anti-HPA-1a antibodies in the sample. The method may further comprise obtaining the sample from the subject.

The quantity of the anti-HPA-1a antibodies in the sample can be used to calculate the pharmacokinetics of the anti-HPA-1a antibodies administered to the subject. For example, samples of the subject may be obtained at multiple time points following administration of the anti-HPA-1a antibodies, and determination of the quantity of the anti-HPA-1a antibodies at those time points can be used to calculate various pharmacokinetic metrics that include, but are not limited to, Cₘₐₓ, Tₘₐₓ AUC, elimination half-life, clearance, etc.

In some embodiments, the capture reagent may comprise a detectable label in accordance with assays of the invention, and measuring the amount of the detection complex comprises measuring the amount of the detectable label.

In some embodiments, the detection complex is formed by the capture reagent bound to the anti-HPA-1a antibodies, and by a detecting reagent that binds to the anti-HPA-1a antibodies. To this end, the methods of the invention may comprise contacting the sample with a capture reagent that binds to anti-HPA-1a antibodies, wherein the capture reagent comprises a modified integrin β₃; contacting the sample with a detecting reagent that binds to the anti-HPA-1a antibodies and forms a detection complex with the capture reagent; and measuring an amount of the detection complex, wherein the amount of the detection complex indicates the quantity of the anti-HPA-1a antibodies in the sample. The detecting reagent may comprise an antibody or antigen-binding fragment thereof that binds to the anti-HPA-1a antibodies in accordance with assays of the invention. The detecting reagent may further comprise a detectable label in accordance with assays of the invention, and measuring the amount of the detection complex comprises measuring the amount of the detectable label.

The capture reagent may be immobilized on a support as described herein. To this end, the methods of the invention may further comprise immobilizing the capture reagent to a support prior to contacting the sample with the capture reagent.

The detectable label may be selected from labels according to assays of the invention. These labels may be measured by detecting signals generated by light scattering, optical absorbance, fluorescence, chemiluminescence, electrochemiluminescence, radioactivity, magnetic fields, etc. To this end, the methods of the invention may include measuring the detectable label through one or more of these techniques.

The sample in the methods of the present invention may comprise a biological fluid such as whole blood or whole blood components including serum and plasma. In some embodiments, the sample is animal serum, including but not limited to human serum, mouse serum, and donkey serum. In some embodiments, the sample is not a biological fluid. For example, the sample can be a composition comprising an anti-HPA1a antibody in a pharmaceutically acceptable carrier.

In some embodiments, the sample may be obtained from a subject who has been administered anti-HPA-1a antibodies. In some embodiments, the sample is obtained from a woman who is HPA-1a negative and had a previous pregnancy in which the fetus was HPA-1a positive. In other embodiments, the sample may be obtained from an HPA-1a negative subject who has been alloimmunized with HPA-1a. In certain embodiments, the sample is obtained from a woman who is HPA-1a negative and is pregnant. In further embodiments, the sample is obtained from a woman who is HPA-1a negative and is carrying an HPA-1a positive fetus.

In some embodiments, the anti-HPA-1a antibodies in the methods of the present invention may be polyclonal antibodies. In certain embodiments, the polyclonal antibodies may be polyclonal anti-HPA-1a antibodies of the gamma isotype, prepared, for example, by pooling plasma of various donors with anti-HPA-1a antibodies (see, e.g., PCT Publication No. WO 2007/078202, which is incorporated herein by reference). Such polyclonal anti-HPA-1a antibodies may be referred to herein as “anti-HPA-1a gamma globulins.”

In other embodiments, the anti-HPA-1a antibodies in the methods of the present invention may be monoclonal antibodies. The monoclonal anti-HPA-1a antibodies may comprise (i) a VH CDR1 that has the amino acid sequence of SEQ ID NO: 6, (ii) a VH CDR2 that has the amino acid sequence of SEQ ID NO: 7, (iii) a VH CDR3 that has the amino acid sequence of SEQ ID NO: 8 (iv) a VL CDR1 that has the amino acid sequence of SEQ ID NO: 9, (v) a VL CDR2 that has the amino acid sequence of SEQ ID NO: 10, and (vi) a VL CDR3 that has the amino acid sequence of SEQ ID NO: 11, in which the CDRs are defined by IMGT. In certain embodiments, the monoclonal anti-HPA-1a antibodies comprise a heavy chain variable region comprising a sequence having a sequence identity to SEQ ID NO: 4 of at least 80%, or at least 85%, or at least 90%, or at least 95%, or at least 98%, or at least 99%; and a light chain variable region comprising a sequence having a sequence identity to SEQ ID NO: 5 of at least 80%, or at least 85%, or at least 90%, or at least 95%, or at least 98%, or at least 99%. In particular embodiments, the monoclonal anti-HPA-1a antibodies comprise a heavy chain variable region comprising SEQ ID NO: 4 and a light chain variable region comprising SEQ ID NO: 5.

In certain embodiments, the monoclonal antibody can be mAb 26.4 (Eksteen et al., 2015; which is incorporated herein by reference).

The methods of the present invention exhibit accuracy, precision, and selectivity for quantifying anti-HPA-1a antibodies and for measuring pharmacokinetics of anti-HPA-1a antibodies. In some embodiments, quantifying anti-HPA-1a antibodies in at least one sample or determining potency of anti-HPA-1a antibodies in at least one sample results in an RE (%) of within about ±25%, preferably within about ±20%. In some embodiments, quantifying anti-HPA-1a antibodies in at least one sample or determining potency of anti-HPA-1a antibodies in at least two samples results in a CV (%) of less than or equal to about 25%, preferably less than or equal to about 20%.

### Methods of Manufacturing

Aspects of the present invention relate to the use of the assays to manufacture a pharmaceutical composition comprising an anti-HPA-1a antibody. The methods comprise (a) admixing an anti-HPA-1a antibody and a pharmaceutically acceptable carrier to prepare a composition; and (b) measuring the potency of the anti-HPA-1a antibody in the composition to determine the concentration of the anti-HPA-1a antibody in the composition. Optionally, the methods may further comprise adjusting the concentration of the anti-HPA-1a antibody in the composition to a specification concentration for a pharmaceutical composition comprising the anti-HPA-1a antibody.

The pharmaceutical composition may comprise a monoclonal anti-HPA-1a antibody. In some embodiments, the monoclonal anti-HPA-1a antibody comprises (i) a VH CDR1 that has the amino acid sequence of SEQ ID NO: 6, (ii) a VH CDR2 that has the amino acid sequence of SEQ ID NO: 7, (iii) a VH CDR3 that has the amino acid sequence of SEQ ID NO: 8 (iv) a VL CDR1 that has the amino acid sequence of SEQ ID NO: 9, (v) a VL CDR2 that has the amino acid sequence of SEQ ID NO: 10, and (vi) a VL CDR3 that has the amino acid sequence of SEQ ID NO: 11, in which the CDRs are defined by IMGT.

In some embodiments, the monoclonal anti-HPA-1a antibody comprises a heavy chain variable region comprising a sequence having a sequence identity to SEQ ID NO: 4 of at least 80%, or at least 85%, or at least 90%, or at least 95%, or at least 98%, or at least 99%, and a light chain variable region comprising a sequence having a sequence identity to SEQ ID NO: 5 of at least 80%, or at least 85%, or at least 90%, or at least 95%, or at least 98%, or at least 99%. In certain embodiments, the monoclonal anti-HPA-1a antibody comprises a heavy chain variable region comprising SEQ ID NO: 4 and a light chain variable region comprising SEQ ID NO: 5.

Alternatively, the pharmaceutical composition may comprise a polyclonal anti-HPA-1a antibody. In some embodiments, the poly clonal anti-HPA-1a antibody is from plasma of one or more HPA-1a-negative subjects immunized with HPA-1a. In certain embodiments, the subject was alloimmunized with HPA-1a, or was immunized with HPA-1a positive platelets, or was immunized with a purified or recombinant preparation HPA-1a antigen. In particular embodiments, the antibodies are cleared of viruses

### Kits

The assays of the present invention can be provided in the form of a kit. Such a kit may be a packaged combination including the elements of:
(a) a capture reagent comprising a modified integrin β₃; and
(b) instructions on how to perform the assay using the capture reagent.

In certain embodiments, the instructions may indicate how to quantify anti-HPA-1a antibodies in a sample using the capture reagent, or how to measure pharmacokinetics of anti-HPA-1a antibodies administered to a subject using the capture reagent.

In some embodiments, the capture reagent may further comprise a detectable label attached to the modified integrin β₃, in accordance with the assays of the present invention.

In further embodiments, the kit may be a packaged combination including the elements of:
(a) a capture reagent comprising a modified integrin β₃; and
(b) a detecting reagent comprising an antibody or antigen-binding fragment thereof that binds to anti-HPA-1a antibodies; and
(c) instructions on how to perform the assay using the capture reagent and the detecting reagent.

In certain embodiments, the instructions may indicate how to quantify anti-HPA-1a antibodies in a sample using the capture reagent and the detecting reagent, or how to measure pharmacokinetics of anti-HPA-1a antibodies administered to a subject using the capture reagent and the detecting reagent.

In some embodiments, the detecting reagent may further comprise a detectable label attached to the antibody or antigen-binding fragment thereof, in accordance with the assays of the present invention.

In embodiments in which the kits includes a detectable label, the detectable label may be an enzyme. In such embodiments, the kit may further comprise substrates and cofactors required by the enzyme, in accordance with the assays of the present invention.

In some embodiments, the kit further comprises a solid support for the capture reagent, which may be provided as a separate element or on which the capture reagents are already immobilized. Hence, the capture reagent in the kit may be immobilized on a solid support, or it may be immobilized on such support that is included with the kit or provided separately from the kit.

The kit may additionally contain the anti-HPA-1a antibody of interest as a standard (e.g., purified antibody of interest), as well as other additives such as stabilizers, washing and incubation buffers, blocking agents, and the like.

The components of the kit may be provided in predetermined quantities and/or ratios, with the relative amounts of the various reagents suitably varied to provide for concentrations in solution of the reagents that substantially maximize the sensitivity of the assay.

### EXAMPLES

Embodiments of the present disclosure can be further defined by reference to the following non-limiting examples. It will be apparent to those skilled in the art that many modifications, both to materials and methods, can be practiced without departing from the scope of the present disclosure.

### Example 1

The cβ₃ capture reagent was prepared by designing a construct in which the βI domain of β₃ (residues Tyr110-Gly349 of SEQ ID NO: 1) is replaced with the al domain of αL integrin (residues Gly110-Lys286 of SEQ ID NO: 2), as essentially described in Zhou et al. (2018).

In brief, DNA encoding the cβ3 protein, having a nucleotide sequence of SEQ ID NO: 12, was synthesized and cloned into a pcDNA3.1/Hygro⁽⁺⁾ expression vector. The full length expression product of SEQ ID NO: 12 has an amino acid sequence of SEQ ID NO: 13.

The DNA construct was transfected into ExpiHEK293F GnT1⁻ cells. The transfected cells were grown as suspension cultures in ExpiHEK medium in a humified incubator with 8% CO₂ at 37° C. After 10 days, cell-culture supernatant was collected. The tagged cβ₃ protein, having an amino acid sequence of SEQ ID NO: 14, was purified from the supernatant over a streptactin column. The tagged protein was then treated with HRV 3C protease to remove the tag (residues Gly626-Ala694 of SEQ ID NO: 14), and treated with Endoglycosidase H to remove the high-mannose type of N-linked glycans. The resulting untagged, deglycosylated cβ₃ protein, now having an amino acid sequence of SEQ ID NO: 2, was purified over a nickel column.

### Example 2

A study was conducted to evaluate and compare the performance of an ELISA assay in accordance with embodiments of the present invention for quantifying anti-HPA-1a antibodies from the following three sources: (1) polyclonal antibodies anti-HPA-1a gamma globulins; (2) monoclonal antibodies mAb 26.4; and (3) polyclonal antibodies of the WHO Standard.

The assay was designed as an antigen capture sandwich ELISA. The capture reagent comprised a modified integrin β₃, and the detecting reagent was mouse anti-human IgG antibody conjugated to HRP. The antibodies were tested in a matrix comprising blocker casein in PBS (“casein-PBS”). A summary of the assay procedure is as follows:

The wells of the assay plate (MAXISORP®) were coated by adding 100 μL of the capture reagent solution (1.0 sg/mL solution prepared my mixing 12.156 mL of IX PBS with 24 μL of modified integrin 133 (concentration 507.5 μg/mL)). The plate was incubated at 4° C. for 12 to 72 hours on an orbital shaker at 100 RPM.

The coated plate was washed with WB-4 buffer, and 300 μL of casein-PBS was added to all wells. The plate was incubated at ambient room temperature for at least one hours on an orbital shaker at 100 RPM.

The plate was washed with WB-4 buffer, and 300 μL of the antibodies (the anti-HPA-1a gamma globulins, mAb 26.4, or the WHO Standard, diluted as described below) were added to the assay plate. The plate was incubated at ambient room temperature for about one hour on an orbital shaker at 100 RPM.

The plate was washed with WB-4 buffer, and 100 μL of the detection reagent solution (prepared by mixing 297 μL casein-PBS and 3 μL of the mouse anti-human IgG antibody conjugated to HRP to create a stock solution, and then mixing 130 μL of the stock solution with 12.870 mL of casein-PBS) was added to all wells of the assay plate. The plate was incubated at ambient room temperature for about one hour on an orbital shaker at 100 RPM.

The plate was washed with WB-4 buffer, and 100 L of TMB substrate solution (prepared by combining equal volumes of TMB Peroxidase Substrate and Peroxidase Substrate Solution B) was added to all wells of the assay plate. The plate was incubated at ambient room temperature.

100 μL of TMB Stop Solution was added to all well of the assay plate. The assay plate was read (Spectra MAX UVNIS with SOFTmax PRO GxP) at 450 nm with reference wavelength at 650 nm.

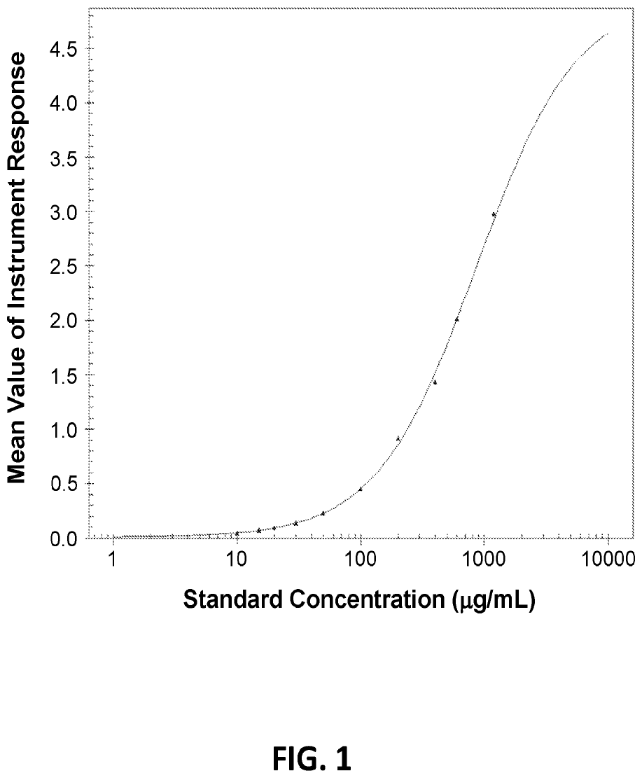

Calibration curves consisting of nine non-zero standards were processed for each of the anti-HPA-1a gamma globulins, mAb 26.4, and the WHO Standard. For the anti-HPA-1a gamma globulins, standards ranging from 15.00 to 750 μg/mL, with accessory standards at 10.00 and 1200.00 mg/mL, were prepared by spiking casein-PBS with appropriate amounts of anti-HPA-1a gamma globulin stock. The results showed that the response correlated well over the concentration range of 15.00 to 750.00 μg/mL in casein-PBS. The measured values for eight out of nine working standards were within +20% relative error (25% for the LLOQ and ULOQ standards) and ≤20% CV. The results for the measured concentrations are presented in Table 1, and a representative calibration curve is shown in FIG. 1.

TABLE 1
Measured concentrations of the anti-HPA-1a gamma
globulins calibration standards in casein-PBS.

| Standard Conc. | | Mean | | |
|---|---|---|---|---|
| (μg/mL) | n | (μg/mL) | RE (%) | % Theoretical |
| 10.00* | 1 | 10.00 | 0.0 | 100.00 |
| 15.00 | 1 | 15.09 | 0.6 | 100.6 |
| 20.00 | 1 | 20.11 | 0.6 | 100.6 |
| 30.00 | 1 | 29.38 | −2.1 | 97.9 |
| 50.00 | 1 | 49.62 | −0.8 | 99.2 |
| 100.00 | 1 | 100.92 | 0.6 | 100.6 |
| 200.00 | 1 | 215.35 | 7.7 | 107.7 |
| 400.00 | 1 | 370.82 | −7.3 | 92.7 |
| 600.00 | 1 | 599.28 | −0.1 | 99.9 |
| 750.00 | 1 | a | a | a |
| 1200.00* | 1 | 1245.67 | 3.8 | 103.8 |

*Accessory standards added to define the lower and upper ends of the calibration curve.
a: outside ± 25% theoretical concentration

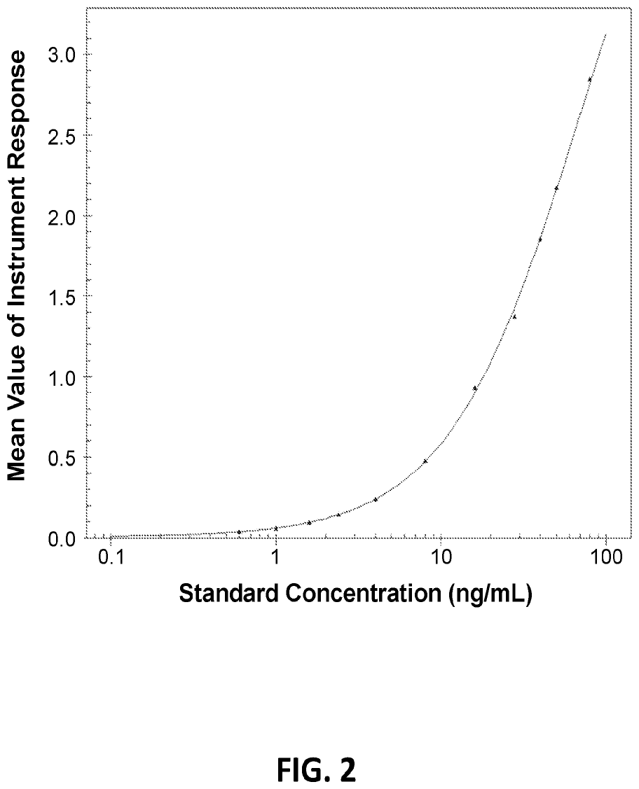

Standards of mAb 26.4 ranging from 1.00 to 50.00 ng/mL, with accessory standards at 0.60 and 80.00 ng/mL, were prepared by spiking casein-PBS with appropriate amounts of mAb 26.4 stock. The results showed that the response correlated well over the entire concentration range, as the measured values for nine out of nine working standards were within ±20% relative error (±25% for the LLOQ and ULOQ standards) and ≤20% CV. The results for the measured concentrations are presented in Table 2, and a representative calibration curve is shown in FIG. 2.

TABLE 2
Measured concentrations of mAb 26.4
calibration standards in casein-PBS.

| Standard Conc. | | Mean | | |
|---|---|---|---|---|
| (ng/mL) | n | (ng/mL) | % RE | % Theoretical |
| 0.60* | 2 | 0.60 | −0.7 | 99.3 |
| 1.00 | 2 | 1.02 | 2.0 | 102.0 |
| 1.60 | 2 | 1.58 | −1.1 | 98.9 |
| 2.40 | 2 | 2.40 | −0.2 | 99.8 |
| 4.00 | 2 | 4.00 | −0.1 | 99.9 |
| 8.00 | 2 | 8.04 | 0.5 | 100.5 |
| 16.00 | 2 | 16.16 | 1.0 | 101.0 |
| 28.00 | 2 | 27.23 | −2.8 | 97.2 |
| 40.00 | 2 | 40.66 | 1.7 | 101.7 |
| 50.00 | 2 | 50.45 | 0.9 | 100.9 |
| 80.00* | 2 | 79.78 | −0.3 | 99.7 |

*Accessory standards added to define the lower and upper ends of the calibration curve.

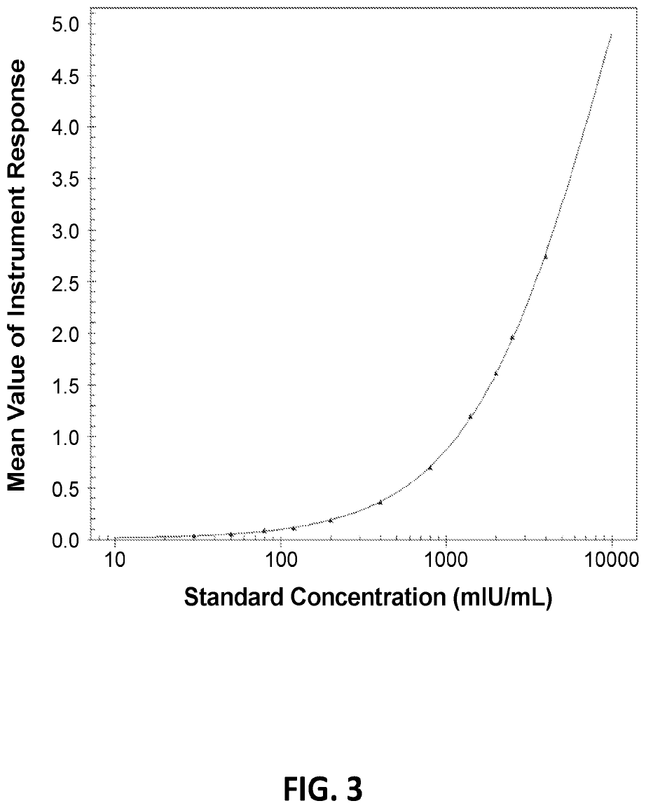

Standards of the WHO Standard ranging from 50.00 to 2500.00 mIU/mL, with accessory standards at 30.00 and 4000.00 mIU/mL, were prepared by spiking casein-PBS with appropriate amounts of WHO Standard stock. The results showed that the response correlated well over the full concentration range, as the measured values for nine out of nine working standards were within ±20% relative error (±25% for the LLOQ and ULOQ standards) and ≤20% CV. The results for the measured concentrations are presented in Table 3, and a representative calibration curve is shown in FIG. 3.

TABLE 3
Measured concentrations of WHO Standard
calibration standards in casein-PBS.

| Standard Conc. | | Mean | | |
|---|---|---|---|---|
| (mIU/mL) | n | (mIU/mL) | RE (%) | % Theoretical |
| 30.00* | 1 | 29.93 | −0.2 | 99.8 |
| 50.00 | 1 | 48.88 | −2.2 | 97.8 |
| 80.00 | 1 | 85.42 | 6.8 | 106.8 |
| 120.00 | 1 | 116.83 | −2.6 | 97.4 |
| 200.00 | 1 | 199.66 | −0.2 | 99.8 |
| 400.00 | 1 | 395.69 | −1.1 | 98.9 |
| 800.00 | 1 | 790.26 | −1.2 | 98.8 |
| 1400.00 | 1 | 1417.60 | 1.3 | 101.3 |
| 2000.00 | 1 | 2010.33 | 0.5 | 100.5 |
| 2500.00 | 1 | 2544.74 | 1.8 | 101.8 |
| 4000.00* | 1 | 3932.89 | −1.7 | 98.3 |

*Accessory standards added to define the lower and upper ends of the calibration curve.

Intra-assay accuracy and precision were also evaluated for each of the anti-HPA-1a gamma globulins, mAb 26.4, and the WHO Standard. Intra-assay accuracy was considered acceptable if % RE was within ±20% (±25% for LLOQ and ULOQ) of their theoretical concentrations, and intra-assay precision was considered acceptable if CV was ≤20% (≤25% for LLOQ and ULOQ).

For the anti-HPA-1a gamma globulins, QC samples were prepared by spiking casein-PBS with appropriate amounts of the anti-HPA-1a gamma globulin stock at concentrations of 15.00 (LLOQ), 30.00 (QC1), 150.00 (QC2), 575.00 (QC3), and 750.00 (ULOQ) μg/mL. As shown in Table 4, intra-assay accuracy and precision were within the acceptable criteria at each concentration.

TABLE 4
Results for intra-assay accuracy and precision for the anti-HPA-1a
gamma globulins at different concentrations in casein-PBS.

| | | | | | | Total |
|---|---|---|---|---|---|---|
| Theoretical | | Mean | | | | Error |
| Conc. (μg/mL) | Replicates | (μg/mL) | SD | % RE | % CV | (%) |
| LLOQ: 15.00 | 6 | 14.28 | 0.85 | −4.8 | 5.9 | 10.7 |
| QC1: 30.00 | 6 | 27.02 | 2.79 | −9.9 | 10.3 | 20.2 |
| QC2: 150.00 | 6 | 134.24 | 9.63 | −10.5 | 7.2 | 17.7 |
| QC3: 575.00 | 6 | 583.02 | 64.50 | 1.4 | 11.1 | 12.5 |
| ULOQ: 750.00 | 6 | 830.49 | 72.29 | 10.7 | 8.7 | 19.4 |

For mAb 26.4, QC samples were prepared by spiking casein-PBS with appropriate amounts of mAb 26.4 stock at concentrations of 1.00 (LLOQ), 2.00 (QC1), 10.00 (QC2), 37.50 (QC3), and 50.00 (ULOQ) ng/mL. Intra-assay accuracy and precision were within the acceptable criteria at each concentration (see Table 5).

TABLE 5
Results for intra-assay accuracy and precision for
mAb 26.4 at different concentrations in casein-PBS.

| | | | | | | Total |
|---|---|---|---|---|---|---|
| Theoretical | | Mean | | | | Error |
| Conc. (ng/mL) | Replicates | (ng/mL) | SD | % RE | % CV | (%) |
| LLOQ: 1.00 | 6 | 1.01 | 0.05 | 1.0 | 5.4 | 6.3 |
| QC1: 2.00 | 6 | 1.91 | 0.18 | −4.3 | 9.3 | 13.6 |
| QC2: 10.00 | 6 | 9.14 | 0.68 | −8.6 | 7.4 | 16.1 |
| QC3: 37.50 | 6 | 39.77 | 3.40 | 6.1 | 8.5 | 14.6 |
| ULOQ: 50.00 | 6 | 52.27 | 5.51 | 4.5 | 10.5 | 15.1 |

For the WHO Standard, QC samples were prepared by spiking casein-PBS with appropriate amounts of the WHO Standard stock at concentrations of 50.00 (LLOQ), 100.00 (QC1), 500.00 (QC2), 1900.00 (QC3), and 2500.00 (ULOQ) mIU/mL. Intra-assay accuracy and precision were within the acceptable criteria at each concentration, as shown in Table 6.

TABLE 6
Results for intra-assay accuracy and precision for the
WHO Standard at different concentrations in casein-PBS.

| Theoretical | | | | | | Total |
|---|---|---|---|---|---|---|
| Conc. | | Mean | | | | Error |
| (mIU/mL) | Replicates | (mIU/mL) | SD | % RE | % CV | (%) |
| LLOQ: 50.00 | 6 | 47.49 | 2.34 | −5.0 | 4.9 | 9.9 |
| QC1: 100.00 | 6 | 96.10 | 1.13 | −3.9 | 1.2 | 5.1 |
| QC2: 500.00 | 6 | 498.29 | 6.44 | −0.3 | 1.3 | 1.6 |
| QC3: 1900.50 | 6 | 1892.13 | 50.31 | −0.4 | 2.7 | 3.1 |
| ULOQ: 2500.00 | 6 | 2548.37 | 63.63 | 1.9 | 2.5 | 4.4 |

The potencies of the anti-HPA-1a gamma globulins and mAb 26.4 were demonstrated by evaluating QC samples containing known amounts of either the anti-HPA-1a gamma globulins or WHO Standard and measured off against an mAb 26.4 standard curve. To determine potency of the anti-HPA-1a gamma globulins, samples were prepared at the anti-HPA-1a gamma globulin concentrations QC1, QC2, and QC3 (theoretical concentrations), diluted from the anti-HPA-1a gamma globulin solution concentration (i.e., 51,000 mIU/mL). The measurement of each sample against the mAb 26.4 standard curve was replicated four times, and the measured concentrations were used to calculate adjusted concentrations as described above. The mean of the adjusted concentrations of the anti-HPA-1a gamma globulins were used to compare the anti-HPA-1a gamma globulins samples to mAb 26.4 and determine the anti-HPA-1a gamma globulins potency to be 3.9 IU/mg (the anti-HPA-1a gamma globulins potency=the anti-HPA-1a gamma globulin mean adjusted concentration/the anti-HPA-1a gamma globulins stock concentration×mAb 26.4 potency) (see Table 7).

TABLE 7
Results of the comparability study of the
anti-HPA-1a gamma globulins with mAb 26.4.

| Theoretical | | Mean | | | |
|---|---|---|---|---|---|
| Conc. | Dilution | Measured | Mean Adjusted | Mean Adjusted | Cumulative |
| (μg/mL) | Fold | Conc. (ng/mL) | Conc. (ng/mL)^{a} | Conc. (ng/mL)^{b} | % CV |
| QC3: 575.00 | 89 | 38.41 | 3406.55 | 3386.80 | 1.9 |
| QC2: 150.00 | 340 | 10.02 | 3407.28 | | |
| QC1: 30.00 | 1700 | 1.97 | 3346.56 | | |

^{a}mean Adjusted Concentration per QC sample
^{b}mean Adjusted Concentration across all QC samples

To determine potency of mAb 26.4, samples were prepared at the WHO Standard QC1, QC2, and QC3 (theoretical concentrations), diluted from the WHO Standard stock solution concentration (i.e., 100,000 mIU/mL). The measurement of each sample against the mAb 26.4 standard curve was replicated four times, and the measured concentrations were used to calculate adjusted concentrations based on the dilution (i.e., adjusted concentration=measured concentrations×dilution fold, in which the dilution fold=stock solution concentration/theoretical concentration) (see Table 8). The mean of the adjusted concentrations of the WHO Standard were used to compare WHO Standard samples to mAb 26.4 and determine mAb 26.4 potency to be 58.7 IU/μg (mAb 26.4 potency=WHO Standard stock solution concentration/WHO Standard mean adjusted concentration).

TABLE 8
Results of the comparability study of WHO Standard with mAb 26.4.

| Theoretical | | Mean | | | |
|---|---|---|---|---|---|
| Conc. | Dilution | Measured | Mean Adjusted | Mean Adjusted | Cumulative |
| (mIU/mL) | Fold | Conc. (ng/mL) | Conc. (ng/mL)^{a} | Conc. (ng/mL)^{b} | % CV |
| QC3: 1900.00 | 53 | 32.66 | 1719.17 | 1704.63 | 2.0 |
| QC2: 500.00 | 200 | 8.54 | 1708.08 | | |
| QC1: 100.00 | 1000 | 1.69 | 16.86.64 | | |

^{a}mean Adjusted Concentration per QC sample
^{b}mean Adjusted Concentration across all QC samples

### Example 3

A validation study was conducted to evaluate the performance of an ELISA assay according to embodiments of the present invention for the determination of the anti-HPA-1a gamma globulins in mouse serum. The study involved assessing how the assay performed as compared to specified acceptance criteria with respect to range of response, intra- and inter-assay precision and accuracy, selectivity, dilution linearity, and prozone assessment. In addition, the stability of test samples at ambient room temperature and/or 4° C., after at least five freeze and thaw cycles in a freezer set to maintain −80° C., and long-term storage in a freezer set to maintain −80° C., was assessed.

The assay was designed as an antigen capture sandwich ELISA. The capture reagent comprised a modified integrin β₃, and the detecting reagent was mouse anti-human IgG antibody conjugated to HRP. A summary of the assay procedure is as follows:

The wells of the assay plate (MAXISORP®) were coated by adding 100 μL of the capture reagent solution (1.0 μg/mL solution prepared my mixing 24 mL of 1×PBS with 47 μL of modified integrin β₃ (concentration 507.5 μg/mL)). The plate was incubated at 4° C. for 12 to 72 hours on an orbital shaker at 100 RPM.

The coated plate was washed with WB-4 buffer, and 300 μL of casein-PBS was added to all wells. The plate was incubated at ambient room temperature for at least one hours on an orbital shaker at 100 RPM.

The plate was washed with WB-4 buffer, and 100 μL of the anti-HPA-1a gamma globulin samples (standards, quality controls, etc., as described below) were added to the assay plate. The plate was incubated at ambient room temperature for about one hour on an orbital shaker at 100 RPM.

The plate was washed with WB-4 buffer, and 100 μL of the detection reagent solution (prepared by mixing 495 μL casein-PBS and 5 μL of the mouse anti-human IgG antibody conjugated to HRP to create a stock solution, and then mixing 240 μL of the stock solution with 23.760 mL of casein-PBS) was added to all wells of the assay plate. The plate was incubated at ambient room temperature for about one hour on an orbital shaker at 100 RPM.

The plate was washed with WB-4 buffer, and 100 μL of TMB substrate solution (prepared by combining equal volumes of TMB Peroxidase Substrate and Peroxidase Substrate Solution B) was added to all wells of the assay plate. The plate was incubated at ambient room temperature.

100 μL of TMB Stop Solution was added to all well of the assay plate. The assay plate was read (Spectra MAX UVNIS with SOFTmax PRO GxP) at 450 nm with reference wavelength at 650 nm.

### Range of Response

A total of 11 calibration curves consisting of nine non-zero standards (assayed as duplicate) were processed. The lower limit of quantitation (LLOQ) and upper limit of quantitation (ULOQ) were defined as the anti-HPA-1a gamma globulin concentrations of the lowest and the highest working standards, respectively, in the calibration curve, and were validated by measuring the LLOQ and ULOQ quality controls (QCs) against the calibration curve and obtaining an acceptable precision and accuracy (see below).

An evaluation was undertaken to determine the simplest regression model and weighting that provides the best fit for the data obtained. The appropriate regression parameters were calculated, and the curve parameters were calculated for each validation run.

The following acceptance criteria were set:
Measured concentrations of standards should be within ±20% (±25% for LLOQ and ULOQ standards) RE (%).
CV (%) of the instrument response for each standard should be 20%.
Curve should have at least six non-zero standards after deletion of calibration points.

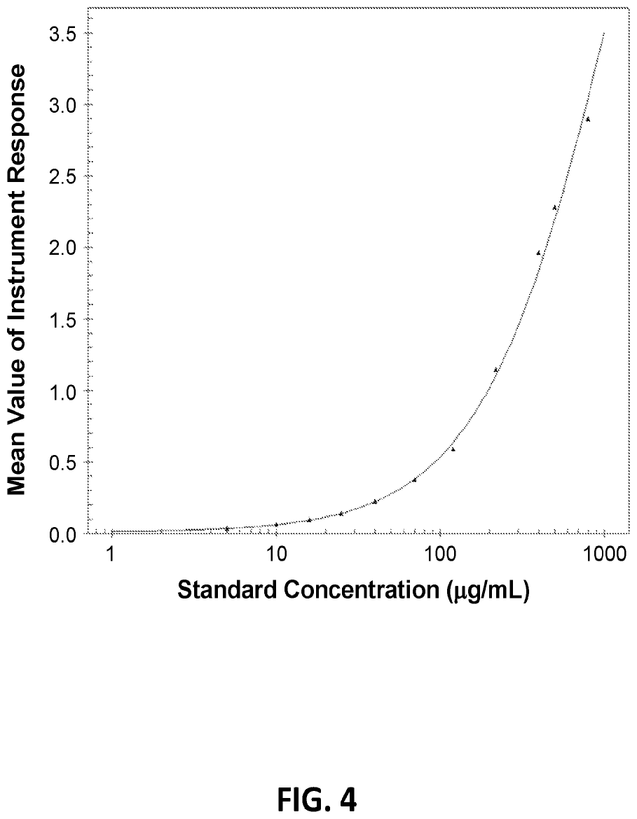

The results showed that the response correlated well over the concentration range of 10.00 to 500.00 μg/mL of the anti-HPA-1a gamma globulins in mouse serum. Accessory standards (additional standards with a concentration either below the lowest working standard or above the highest working standard that was used to define the lower and the upper ends of the calibration curve) at 5.00 and 800.00 μg/mL were added. The measured values for at least eight out of nine standards were within +20% RE (%) and ≤20% CV (%). The results for the measured anti-HPA-1a gamma globulin concentrations and a representative calibration curve are presented in Table 9 and FIG. 4, respectively.

TABLE 9
Measured Concentrations of the anti-HPA-1a gamma
globulin calibration standards in mouse serum.

| Standard Conc. | | Mean | | | |
|---|---|---|---|---|---|
| (μg/mL) | n | (μg/mL) | SD | RE (%) | CV (%) |
| 5.00* | 9 | 5.04 | 0.17 | 0.8 | 3.4 |
| 10.00 | 8 | 9.99 | 0.41 | −0.1 | 4.1 |
| 16.00 | 10 | 15.83 | 0.24 | −1.1 | 1.5 |
| 25.00 | 10 | 25.22 | 0.78 | 0.9 | 3.1 |
| 40.00 | 10 | 40.43 | 1.40 | 1.1 | 3.5 |
| 70.00 | 10 | 70.01 | 1.92 | 0.1 | 2.7 |
| 120.00 | 10 | 119.00 | 5.62 | −0.8 | 4.7 |
| 220.00 | 10 | 220.52 | 7.12 | 0.2 | 3.2 |
| 400.00 | 10 | 399.91 | 17.55 | −0.1 | 4.4 |
| 500.00 | 10 | 511.79 | 14.36 | 2.4 | 2.8 |
| 800.00* | 10 | 792.31 | 32.31 | −1.0 | 4.1 |

*Accessory standards added to define the lower and upper ends of the calibration curve.

### Inter- and Intra-Assay Precision and Accuracy

QC samples of the anti-HPA-1a gamma globulins in mouse serum were assayed in four replicates on six separate occasions (i.e., analytical runs) at the following five anti-HPA-1a gamma globulin concentrations: 10.00 μg/mL (LLOQ), 30.00 μg/mL (QC1, which was set for three times the LLOQ), 100.00 μg/mL (QC2, which was considered a scientifically appropriate concentration based on the mid-point of the assay), 375.00 μg/mL (QC3, which was 75% of the ULOQ), and 500.00 μg/mL (ULOQ). Measured concentrations for the QC samples were determined by the curve fitting regression program generated from the calibration standards. For each QC concentration, mean and standard deviation were calculated for the measured concentrations.

The intra- and inter-assay accuracy were considered acceptable if the RE (%) was within ±20% (±25% for LLOQ and ULOQ) of the theoretical concentration at each concentration. The intra- and inter-assay precision was considered acceptable if the CV (%) was ≤20% (≤25% for LLOQ and ULOQ). In addition, the total error (%) (calculated as the sum of CV (%) and absolute RE (%)) should be ≤30% (≤40% for LLOQ and ULOQ).

The results, summarized in Tables 10 and 11, showed that the intra- and inter-assay accuracy and precision were all within the acceptance criteria.

TABLE 10
Intra-assay precision and accuracy of the
anti-HPA-1a gamma globulins in mouse serum.

| Theoretical | | | | |
|---|---|---|---|---|
| Conc. | Analytical | | RE (%) | CV (%) |
| (μg/mL) | Runs | Replicates | (range) | (range) |
| LLOQ: 10.00 | 6 | 4 | −12.7 to 0.3 | 0.9 to 9.4 |
| QC1: 30.00 | 6 | 4 | −8.5 to 1.4 | 1.9 to 6.8 |
| QC2: 100.00 | 6 | 4 | −11.1 to 7.3 | 1.4 to 8.4 |
| QC3: 375.00 | 6 | 4 | −7.5 to 4.9 | 0.9 to 8.0 |
| ULOQ: 500.00 | 6 | 4 | −1.8 to 6.5 | 2.2 to 7.8 |

TABLE 11
Inter-assay precision and accuracy of the
anti-HPA-1a gamma globulins in mouse serum.

| | | | | | | Total |
|---|---|---|---|---|---|---|
| Theoretical | | Mean | | RE | CV | Error |
| Conc. (μg/mL) | n | (μg/mL) | SD | (%) | (%) | (%) |
| LLOQ: 10.00 | 18 | 9.64 | 0.54 | −3.6 | −3.6 | 9.2 |
| QC1: 30.00 | 17 | 29.32 | 1.44 | −2.3 | −2.3 | 7.2 |
| QC2: 100.00 | 18 | 98.23 | 7.05 | −1.8 | −1.8 | 9.0 |
| QC3: 375.00 | 18 | 371.24 | 23.79 | −1.0 | −1.0 | 7.4 |
| ULOQ: 500.00 | 17 | 513.46 | 25.31 | 2.7 | 2.7 | 7.6 |

### Selectivity: Normal Mouse Serum

Mouse serum from ten different animals were analyzed both without and with the anti-HPA-1a gamma globulins (unspiked and spiked, respectively) at the concentration targeting the LLOQ and high QC (QC3) in order to check for interference. The interference check was performed to ensure that the assay was specific for the anti-HPA-1a gamma globulins and can select the anti-HPA-1a gamma globulins from a complex matrix without positive or negative interference.

Selectivity was accepted if at least 80% of the unspiked mouse serum lots tested were below the LLOQ and if at least 80% of the spiked mouse serum samples at each concentration had recoveries within ±20% relative error at the QC3 level and ±25% relative error at the LLOQ level and CV (%) is ≤20% for each replicate.

All tested unspiked samples gave concentration results below the LLOQ of 10.00 μg/mL. All tested spike samples gave recoveries within ±25% RE (%) with CV (%)≤20% for each replicate at the 10.00 (LLOQ) μg/mL level, and all tested spike samples gave recoveries within ±25% RE (%) with CV (%)≤20% for each replicate at the 375.00 μg/mL (high) level. The results of the selectivity analysis are presented in Table 12.

TABLE 12
Selectivity of the anti-HPA-1a gamma globulins in mouse serum.

| | Unspiked | Spiked | | Spiked | |
|---|---|---|---|---|---|
| | Serum | Serum | | Serum | |
| Sample | (μg/mL) | (10.00 μg/mL) | RE (%) | (375.00 μg/mL) | % RE |
| 1 | <LLOQ | 9.73 | −2.7 | 373.72 | −0.3 |
| 2 | <LLOQ | 10.07 | 0.7 | 380.29 | 1.4 |
| 3 | <LLOQ | 10.15 | 1.5 | 360.49 | −3.9 |
| 4 | <LLOQ | 10.00 | 0.0 | 378.87 | 1.0 |
| 5 | <LLOQ | 10.30 | 3.0 | 395.10 | 5.4 |
| 6 | <LLOQ | 9.82 | −1.8 | 380.70 | 1.5 |
| 7 | <LLOQ | 10.08 | 0.8 | 409.33 | 9.2 |
| 8 | <LLOQ | 10.01 | 0.1 | 357.31 | −4.7 |
| 9 | <LLOQ | 9.60 | −4.0 | 376.30 | 0.3 |
| 10 | <LLOQ | 10.15 | 1.5 | 396.30 | 5.7 |

### Selectivity: 5% Hemolyzed Mouse Serum

Mouse serum hemolyzed whole blood (100%) from one animal was diluted with blank matrix (preferably from the same animal as the whole blood) to yield 5% v/v hemolyzed matrix. The lot of hemolyzed mouse serum (5%) was analyzed both without and with the anti-HPA-1a gamma globulins (unspiked and spiked, respectively) at the concentration targeting the LLOQ and high QC (QC3) in three replicates in order to check for interference.

Selectivity in 5% (v/v) hemolyzed mouse serum was accepted if the unspiked mouse serum lot tested was below the LLOQ and if the mean concentration of each of the spiked hemolyzed mouse serum sample was within ±20% RE (%) at the QC3 level and within ±25% RE (%) at the LLOQ level and ≤20% CV.

The tested unspiked sample gave a concentration result below the LLOQ of 10.00 μg/mL. The mean concentration of the spiked lot tested gave recoveries within ±25% RE (%) with CV (%)≤20% at the 10.00 (LLOQ) μg/mL level, and within ±20% RE (%) with CV (%)≤20% at the 375.00 (high) μg/mL level. The results of the selectivity analysis are presented in Table 13.

TABLE 13
Selectivity of the anti-HPA-1a gamma globulins
in 5% (v/v) hemolyzed mouse serum.

| Theoretical | | | | | |
|---|---|---|---|---|---|
| Conc | | Mean | | | |
| (μg/mL) | n | (μg/mL) | SD | RE (%) | CV (%) |
| Unspiked Serum | 3 | <LLOQ | N/A | N/A | N/A |
| Spiked Serum | 3 | 9.91 | 0.55 | −0.9 | 5.6 |
| (10.00 μg/mL) | | | | | |
| Spiked Serum | 3 | 382.81 | 6.86 | 2.1 | 1.8 |
| (375.00 μg/mL) | | | | | |

### Prozone Effect and Dilution Linearity

Prozone effect was assessed in over range stocks spiked with the anti-HPA-1a gamma globulins in mouse serum. The highest concentration exceeded the highest concentration predicted during corresponding sample analysis studies (if feasible while maintaining at least a 90% proportion of mouse serum). The high concentration sample was also diluted with suitable mouse serum down to two additional over range stocks (2× the ULOQ and the geometrical mid-point between the high and low prozone concentrations for example). The lowest prozone concentration was diluted to at least one concentration within the curve range. Over-range stocks for prozone assessment and the in-range sample were analyzed at n=3 duplicates. The mean back-calculated concentrations of each prozone sample should be >ULOQ.

Dilution linearity was assessed in over range stocks spiked with the anti-HPA-1a gamma globulins in mouse serum and diluted into the curve range. The highest concentration exceeded the highest concentration predicted during corresponding sample analysis studies (if feasible while maintaining at least a 90% proportion of mouse serum). Lower concentration dilution linearity samples was prepared at appropriate concentrations (at least two additional concentrations) to cover the range of dilutions anticipated in future sample analysis studies. The lowest concentration dilution linearity sample was serially diluted to three concentrations within the dynamic range of the assay. The higher concentration dilution linearity samples should be diluted to at least one concentration within the dynamic range of the assay. Dilution linearity dilutions were assessed in a total of at least six replicates (six separate dilutions from each linearity sample). Dilution linearity accuracy and precision were calculated on adjusted values (i.e., measured concentration multiplied by dilution factor). To be considered acceptable, each dilution factor tested was to have a mean relative error±20% and 20% CV (%) and at least two thirds of replicates must meet the relative error criterion. The cumulative precision of all adjusted values derived from a single linearity sample was to be within 20% CV (%).

The results from the prozone effect evaluation gave measured concentrations that were above the ULOQ (500.00 μg/mL) at each concentration. As a result, a prozone effect was not observed. The results from the dilution linearity analysis indicated that high concentration samples of the anti-HPA-1a gamma globulins in mouse serum could be diluted within the range of the standard curve up to 50-fold with blank mouse serum. The dilution linearity results are presented in Table 14 and Table 15.

TABLE 14
Dilution linearity for each replicate of the
anti-HPA-1a gamma globulins in mouse serum.

| | | Measured | Adjusted | | | |
|---|---|---|---|---|---|---|
| | | Conc. | Conc. | RE | | CV |
| Sample | Replicate | (μg/mL) | (μg/mL) | (%) | SD | (%) |
| Initial Conc.: 5000.00 μg/mL | 1 | 106.65 | 5332.42 | 6.6 | 5.84 | 5.8 |
| Dilution (fold): 50 | 2 | 105.31 | 5265.42 | 5.3 | | |
| Theoretical Conc.: 100.00 μg/mL | 3 | 91.73 | 4586.44 | −8.3 | | |
| | 4 | 102.36 | 5118.15 | 2.4 | | |
| | 5 | 95.62 | 4781.22 | −4.4 | | |
| | 6 | 97.76 | 4887.93 | −2.2 | | |
| Initial Conc.: 2500.00 μg/mL | 1 | 103.53 | 2588.32 | 3.5 | 4.50 | 4.7 |
| Dilution (fold): 25 | 2 | 97.19 | 2429.82 | −2.8 | | |
| Theoretical Conc.: 100.00 μg/mL | 3 | 89.40 | 2234.96 | −10.6 | | |
| | 4 | 96.46 | 2411.61 | −3.5 | | |
| | 5 | 96.45 | 2411.33 | −3.5 | | |
| | 6 | 95.50 | 2387.51 | −4.5 | | |
| Initial Conc.: 1000.00 μg/mL | 1 | 403.85 | 1009.61 | 1.0 | 25.73 | 6.4 |
| Dilution (fold): 2.5 | 2 | 402.77 | 1006.92 | 0.7 | | |
| Theoretical Conc.: 400.00 μg/mL | 3 | 445.63 | 1114.07 | 11.4 | | |
| | 4 | 410.61 | 1026.52 | 2.7 | | |
| | 5 | 371.63 | 929.08 | −7.1 | | |
| | 6 | 381.76 | 954.39 | −4.6 | | |
| Initial Conc.: 1000.00 μg/mL | 1 | 88.34 | 883.39 | −11.7 | 3.74 | 4.0 |
| Dilution (fold): 10 | 2 | 91.01 | 910.05 | −9.0 | | |
| Theoretical Conc.: 100.00 μg/mL | 3 | 95.50 | 955.01 | −4.5 | | |
| | 4 | 97.31 | 973.15 | −2.7 | | |
| | 5 | 93.04 | 930.44 | −7.0 | | |
| | 6 | 97.84 | 978.45 | −2.2 | | |
| Initial Conc.: 1000.00 μg/mL | 1 | 20.70 | 1034.90 | 3.5 | 0.33 | 1.6 |
| Dilution (fold): 50 | 2 | 20.40 | 1019.94 | 2.0 | | |
| Theoretical Conc.: 20.00 μg/mL | 3 | 20.13 | 1006.46 | 0.6 | | |
| | 4 | 19.76 | 988.04 | −1.2 | | |
| | 5 | 20.03 | 1001.60 | 0.2 | | |
| | 6 | 20.40 | 1019.95 | 2.0 | | |

TABLE 15
Dilution linearity in total of the anti-HPA-1a gamma globulins in mouse serum.

| | Mean Conc. | Final Conc. | | Cumulative |
|---|---|---|---|---|
| Sample | (μg/mL) | (μg/mL) | RE (%) | CV (%) |
| Initial Conc.: 5000.00 μg/mL | 99.91 | 4995.26 | −0.1 | N/A |
| Dilution (fold): 50 | | | | |
| Theoretical Conc.: 100.00 μg/mL | | | | |
| Initial Conc.: 2500.00 μg/mL | 96.42 | 2410.59 | −3.6 | N/A |
| Dilution (fold): 25 | | | | |
| Theoretical Conc.: 100.00 μg/mL | | | | |
| Initial Conc.: 1000.00 μg/mL | 402.71 | 1006.77 | 0.7 | 5.5 |
| Dilution (fold): 2.5 | | | | |
| Theoretical Conc.: 400.00 μg/mL | | | | |
| Initial Conc.: 1000.00 μg/mL | 93.84 | 938.41 | −6.2 | |
| Dilution (fold): 10 | | | | |
| Theoretical Conc.: 100.00 μg/mL | | | | |
| Initial Conc.: 1000.00 μg/mL | 20.24 | 1011.82 | 1.2 | |
| Dilution (fold): 50 | | | | |
| Theoretical Conc.: 20.00 μg/mL | | | | |

### Storage Stability

For evaluating short-term stability, aliquots of each of the anti-HPA-1a gamma globulin concentrations QC1 and QC3 were stored at ambient room temperature and/or in a refrigerator set to maintain 4° C. for at least 12 hours, dependent on the expected duration that samples will be maintained at these conditions. The short-term stability was evaluated (n=3) by comparing the stability samples with their theoretical concentrations. Short-term stability samples were analyzed using a freshly prepared standard curve. Samples designated for use as short-term stability samples were shown to meet acceptance criteria (target±10% of theoretical concentration) prior to storage. Short term stability was considered acceptable if the mean concentration of the stability samples was within ±20% RE (%) and ≤20% CV (%) at each concentration.

For evaluating freeze-thaw matrix stability, testing for freeze and thaw stability were determined for a minimum of five freeze and thaw cycles. Aliquots of each of the anti-HPA-1a gamma globulin concentrations QC1 and QC3 were stored in a freezer set to maintain −80° C. for at least 24 hours and completely thawed unassisted at ambient room temperature and/or in a refrigerator set to maintain 4° C. for at least one hour. The cycle of freezing (for at least 12 hours) and thawing was repeated at least four more times, followed by analysis. The freeze-thaw stability was evaluated (n=3) by comparing the stability samples with their theoretical concentrations. Freeze-thaw stability samples were analyzed using a freshly prepared standard curve. Freeze-thaw stability was considered acceptable if the mean concentration of the stability samples was within ±20% RE (%) and ≤20% CV (%) at each concentration.

For evaluating long-term freezing storage stability that reflect the storage of the test samples, aliquots (n=3) were evaluated in a freezer set to maintain −80° C. for a duration to cover the anticipated storage period of the test samples by comparing the stability samples with their theoretical concentration. The stability samples were the QC1 and QC3 that have been stored in a freezer set to maintain −80° C. for the appropriate time duration. Samples designated for use as long-term stability assessment samples were shown to meet acceptance criteria (target±10% of theoretical concentration) prior to storage. Long-term stability samples were analyzed using a freshly prepared standard curve. Stability was considered acceptable if the mean concentration of the stability samples was within ±20% RE (%) and ≤20% CV (%) at each concentration.

The results show that the anti-HPA-1a gamma globulins in mouse serum were stable for 20 hours at ambient room temperature and in a refrigerator set to maintain 4° C., after five freeze-thaw cycles in a freezer set to maintain −80° C. and up to 109 days in a freezer set to maintain −80° C. The results are summarized in Table 16.

TABLE 16
Stability of the anti-HPA-1a gamma globulins
in mouse serum at various storage conditions.

| | | | | | |
|---|---|---|---|---|---|
| | | Mean | | | |
| Samples | n | (μg/mL) | SD | CV (%) | RE (%) |

Storage for 19 Hours, 53 Minutes at Ambient Room Temperature

| | | | | | |
|---|---|---|---|---|---|
| QC1: 30.00 μg/mL | 3 | 35.24 | 1.51 | 4.3 | 17.5 |
| QC3: 375.00 μg/mL | 3 | 467.21 | 46.67 | 10.0 | 24.6 |

Storage for 20 Hours, 10 Minutes at Ambient Room Temperature

| | | | | | |
|---|---|---|---|---|---|
| QC1: 30.00 μg/mL | 3 | 29.81 | 0.35 | 1.2 | −0.6 |
| QC3: 375.00 μg/mL | 3 | 375.24 | 25.96 | 6.9 | 0.1 |

Storage for 20 Hours, 14 Minutes at 4° C.

| | | | | | |
|---|---|---|---|---|---|
| QC1: 30.00 μg/mL | 3 | 29.03 | 2.10 | 7.2 | −3.2 |
| QC3: 375.00 μg/mL | 3 | 353.28 | 0.64 | 0.2 | −5.8 |

After Five Freeze-Thaw Cycles at −80° C.

| | | | | | |
|---|---|---|---|---|---|
| QC1: 30.00 μg/mL | 3 | 30.24 | 0.57 | 1.9 | 0.8 |
| QC3: 375.00 μg/mL | 3 | 346.25 | 15.73 | 4.5 | −7.7 |

Storage for 109 Days at −80° C.

| | | | | | |
|---|---|---|---|---|---|
| QC1: 30.00 μg/mL | 3 | 28.10 | 0.87 | 3.1 | −6.3 |
| QC3: 375.00 μg/mL | 3 | 391.54 | 30.28 | 7.7 | 4.4 |

### Conclusion

The validation data demonstrated that the assay was suitable for the analysis of the anti-HPA-1a gamma globulins in mouse serum. The selectivity data met the acceptance criteria described for this method, indicating that the method was selective for the anti-HPA-1a gamma globulins. The overall accuracy and precision results were acceptable using a calibration curve range from 10.00 to 500.00 μg/mL. In addition, dilution linearity was established up to 50-fold with no prozone effect observed with this method at 5000.00 μg/mL of the antibodies. The results indicated that the anti-HPA-1a gamma globulins in mouse serum was stable after 5 freeze-thaw cycles in a freezer set to maintain −80° C., at ambient room temperature and in a refrigerator set to maintain 4° C. for 20 hours, and up to 109 days in a freezer set to maintain −80° C.

### Example 4

A study was conducted to evaluate the performance of an ELISA assay according to embodiments of the present invention for the determination of monoclonal anti-HPA-1a antibodies mAb 26.4 in mouse serum. The study involved assessing how the assay performed as compared to specified acceptance criteria with respect to intra-assay accuracy and precision.

The assay was designed as an antigen capture sandwich ELISA. The capture reagent comprised a modified integrin β₃, and the detecting reagent was mouse anti-human IgG antibody conjugated to HRP. The antibodies were tested in a matrix comprising mouse serum in 3% bovine serum albumin (BSA). A summary of the assay procedure is as follows:

The wells of the assay plate (MAXISORP®) were coated by adding the capture reagent solution (0.5 μg/mL of modified integrin 133 and 2 μg/mL of BSA in 1×PBS). The plate was incubated at 4° C. overnight on an orbital shaker at 100 RPM.

The coated plate was washed with WB-4 buffer, and 3% BSA was added to all wells. The plate was incubated at ambient room temperature on an orbital shaker at 100 RPM.

The plate was washed with WB-4 buffer, and 100 μL of mAb 26.4 (in mouse serum and 3% BSA, diluted as described below) were added to the assay plate. The plate was incubated at ambient room temperature on an orbital shaker at 100 RPM.

The plate was washed with WB-4 buffer, and the detection reagent solution (mouse anti-human IgG antibody conjugated to HRP, diluted to 1:10000, prepared in 3% BSA) was added to all wells of the assay plate. The plate was incubated at ambient room temperature on an orbital shaker at 100 RPM.

The plate was washed with WB-4 buffer, and TMB substrate solution (prepared by combining equal volumes of TMB Peroxidase Substrate and Peroxidase Substrate Solution B) was added to all wells of the assay plate. The plate was incubated at ambient room temperature.

TMB Stop Solution was added to all well of the assay plate. The assay plate was read (Spectra MAX UVNIS with SOFTmax PRO GxP) at 450 nm with reference wavelength at 650 nm.

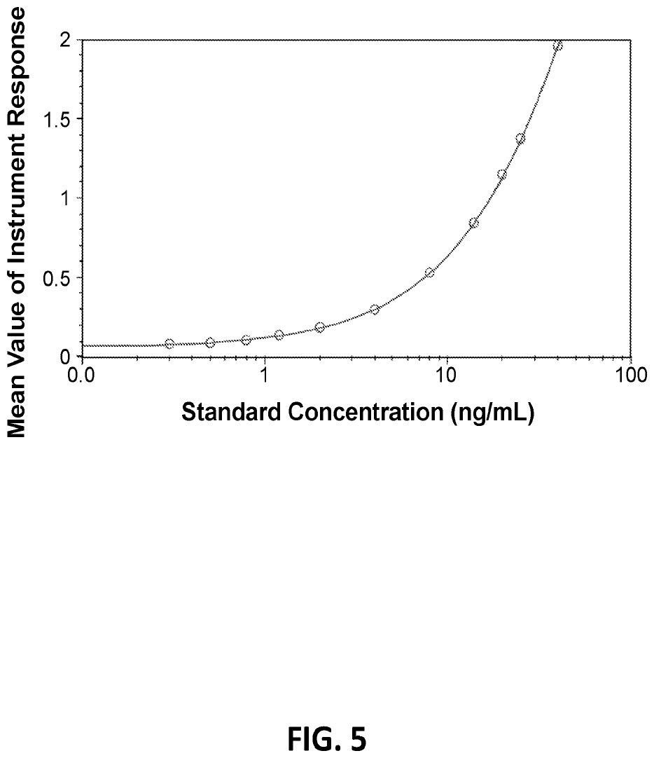

A total of 11 calibration curves consisting of nine non-zero standards (assayed as duplicate) were processed. Standards ranging from 0.50 to 25.00 ng/mL, with accessory standards at 0.30 and 40.00 ng/mL, were prepared. The results showed that the response correlated well over the concentration range of 0.50 to 25.00 ng/mL. The measured values for nine out of nine working standards were within +20% relative error (+25% for the LLOQ and ULOQ standards). The results for the measured concentrations are presented in Table 17, and a representative calibration curve is shown in FIG. 5.

TABLE 17
Measured concentrations of mAb 26.4
calibration standards in mouse serum.

| Standard Conc | | Mean | | |
|---|---|---|---|---|
| (ng/mL) | n | (ng/mL) | RE (%) | % Theoretical |
| 0.30* | 2 | 0.31 | 3.3 | 103.3 |
| 0.50 | 2 | 0.49 | −2.0 | 98.0 |
| 0.80 | 2 | 0.75 | −6.3 | 93.8 |
| 1.20 | 2 | 1.26 | 5.0 | 105.0 |
| 2.00 | 2 | 2.01 | 0.5 | 100.5 |
| 4.00 | 2 | 3.96 | −1.0 | 99.0 |
| 8.00 | 2 | 8.01 | 0.1 | 100.1 |
| 14.00 | 2 | 13.92 | −0.6 | 99.4 |
| 20.00 | 2 | 20.10 | 0.5 | 100.5 |
| 25.00 | 2 | 25.09 | 0.4 | 100.4 |
| 40.00* | 2 | 39.89 | −0.3 | 99.7 |

*Accessory standards added to define the lower and upper ends of the calibration curve.

Intra-assay accuracy and precision were also evaluated. Intra-assay accuracy was considered acceptable if RE (%) was within ±20% (±25% for LLOQ and ULOQ) of their theoretical concentrations, and intra-assay precision was considered acceptable if CV (%) was ≤20% (≤25% for LLOQ and ULOQ).

QC samples were prepared at mAb 26.4 concentrations of 0.50 (LLOQ), 1.50 (QC1), 5.00 (QC2), 19.00 (QC3), and 25.00 (ULOQ) ng/mL. As shown in Table 18, intra-assay accuracy and precision were within the acceptable criteria at each concentration except LLOQ.

TABLE 18
Intra-assay precision and accuracy of mAb 26.4 in mouse serum.

| Theoretical | | | | |
|---|---|---|---|---|
| Conc. | Analytical | | RE (%) | CV (%) |
| (ng/mL) | Runs | Replicates | (range) | (range) |
| LLOQ: 0.50 | 6 | 2 | −6.0 to 32.0 | 0.1 to 7.4 |
| QC1: 1.50 | 6 | 2 | 0.7 to 10.7 | 0.0 to 4.0 |
| QC2: 5.00 | 6 | 2 | 3.4 to 7.2 | 0.4 to 1.6 |
| QC3: 19.00 | 6 | 2 | 2.7 to 10.8 | 0.3 to 2.9 |
| ULOQ: 25.00 | 6 | 2 | 0.8 to 2.8 | 0.8 to 2.8 |

### Example 5

A validation study is conducted to evaluate the performance of an ELISA assay according to embodiments of the present invention for the determination of monoclonal anti-HPA-1a antibodies mAb 26.4 in a vehicle comprising 20 mM histidine, 150 mM arginine, and 0.02% polysorbate 80, at a of pH 6.0. The validation is with respect to range of response, and intra- and inter-assay precision and accuracy. The stability of samples at ambient room temperature and/or 4° C., after at least five freeze and thaw cycles in a freezer set to maintain −80° C., and long-term storage in a freezer set to maintain −80° C., is also assessed. Each of these parameters are evaluated in comparison to specified acceptance criteria.

The assay was designed as an antigen capture sandwich ELISA. The capture reagent comprised a modified integrin β₃, and the detecting reagent was mouse anti-human IgG antibody conjugated to HRP. A summary of the assay procedure is as follows:

The wells of the assay plate (MAXISORP®) are coated by adding 100 μL of the capture reagent solution (1.0 μg/mL solution prepared my mixing 24 mL of 1×PBS with 47 μL of modified integrin β₃ (concentration 507.5 μg/mL)). The plate is incubated at 4° C. for 12 to 72 hours on an orbital shaker at 100 RPM.

The coated plate is washed with WB-4 buffer, and 300 μL of casein-PBS is added to all wells. The plate is incubated at ambient room temperature for at least one hours on an orbital shaker at 100 RPM.

The plate is washed with WB-4 buffer, and 100 μL of mAb 26.4 samples (standards, quality controls, etc., as described below) are added to the assay plate. The plate is incubated at ambient room temperature for about one hour on an orbital shaker at 100 RPM.

The plate is washed with WB-4 buffer, and 100 μL of the detection reagent solution (prepared by mixing 495 μL casein-PBS and 5 μL of the mouse anti-human IgG antibody conjugated to HRP to create a stock solution, and then mixing 240 μL of the stock solution with 23.760 mL of casein-PBS) is added to all wells of the assay plate. The plate is incubated at ambient room temperature for about one hour on an orbital shaker at 100 RPM.

The plate is washed with WB-4 buffer, and 100 μL of TMB substrate solution (prepared by combining equal volumes of TMB Peroxidase Substrate and Peroxidase Substrate Solution B) is added to all wells of the assay plate. The plate is incubated at ambient room temperature.

100 μL of TMB Stop Solution is added to all well of the assay plate. The assay plate is read (Spectra MAX UVNIS with SOFTmax PRO GxP) at 450 nm with reference wavelength at 650 nm.

### Range of Response

A minimum of three calibration curves consisting of at least nine non-zero standards (assayed as duplicate) is processed. The LLOQ and ULOQ are validated by measuring the LLOQ and ULOQ QCs against the calibration curve and obtaining an acceptable precision and accuracy. An evaluation is undertaken to determine the simplest regression model and weighting that provides the best fit for the data obtained. The appropriate regression parameters are calculated, and the curve parameters are calculated for each validation run.

The following acceptance criteria are set:
Measured concentrations of standards should be within ±20% (±25% for LLOQ and ULOQ standards) RE (%).
CV (%) of the instrument response for each standard should be ≤20%.
Curve should have at least seven non-zero standards after deletion of calibration points.

### Inter- and Intra-Assay Precision and Accuracy

QC samples in the vehicle are assayed in at least replicates of three on at least three separate occasions at a minimum of five mAb 26.4 concentrations: LLOQ, low QC (QC1, three times the LLOQ), medium QC (QC2, scientifically appropriate concentration based on the mid-point of the assay), high QC (QC3, 75% of the ULOQ), and ULOQ. Measured concentrations for the QC samples are determined by the curve fitting regression program generated from the calibration standards. For each QC concentration, mean and standard deviation are calculated for the measured concentrations.

The intra- and inter-assay accuracy are considered acceptable if the RE (%) is within ±20% (±25% for LLOQ and ULOQ) of the theoretical concentration at each concentration. The intra- and inter-assay precision are considered acceptable if the CV (%) is ≤20% (≤25% for LLOQ and ULOQ). In addition, the total error (%) (calculated as the sum of CV (%) and absolute RE (%)) should be ≤30% (≤40% for LLOQ and ULOQ).

### Prozone Effect

Prozone effect is assessed in over range stocks spiked with mAb 26.4 in the vehicle. The high concentration sample is diluted with the suitable vehicle down to two additional over range stocks (2× the ULOQ and 10× the ULOQ for example). The lowest prozone concentration is also diluted to at least one concentration within the curve range. Over-range stocks for prozone assessment and the in-range sample should be prepared in one or more replicates and analyzed at n=3 duplicates.

The mean back-calculated concentrations of each prozone sample should be >ULOQ. The in-range sample should have a mean relative error of ±20% and CV between the three replicates of within 20% for acceptance of prozone assessment results.

### Storage Stability

For evaluating short-term stability, aliquots of each of the mAb 26.4 QC1 and QC3 in vehicle are stored at ambient room temperature for at least 16 hours, dependent on the expected duration that samples are maintained at these conditions. The short-term stability is evaluated (n=3 individual aliquots) by comparing the stability samples with their theoretical concentrations. Short-term stability samples are analyzed using a freshly prepared standard curve and assay acceptance QC samples. Samples designated for use as short-term stability samples should be shown to meet acceptance criteria (aiming for ±10% mean relative error) prior to storage. Should short-term stability not meet acceptance criteria, it is repeated for a shorter duration. Short term stability is considered acceptable if the mean concentration of the stability samples is within ±20% RE (%) and ≤20% CV (%) at each concentration.

For evaluating freeze-thaw matrix stability, testing for freeze and thaw stability is determined for a minimum of five freeze and thaw cycles. Aliquots of each of the mAb 26.4 QC1 and QC3 in vehicle is stored in a freezer set to maintain −80° C. for at least 24 hours and completely thawed unassisted at ambient room temperature for at least one hour. The cycle of freezing (for at least 12 hours) and thawing is repeated at least four more times, followed by analysis. The freeze-thaw stability is evaluated (n=3 individual aliquots) by comparing the stability samples with their theoretical concentrations. Freeze-thaw stability samples are analyzed using a freshly prepared standard curve and assay acceptance QC samples. Samples designated for use as freeze-thaw stability samples are shown to meet acceptance criteria (aiming for 10% mean relative error) prior to storage. If considered suitable for storage, this sample is stored neat for future stability testing. If stability after five freeze and thaw cycles does not meet acceptance criteria, stability after four freeze and thaw cycles are assessed. Freeze-thaw stability is considered acceptable if the mean concentration of the stability samples is within ±20% RE (%) and ≤20% CV (%) at each concentration.

For evaluating long-term freezing storage stability, aliquots (n=3) are stored in a freezer set to maintain −80° C. for a duration to cover the anticipated storage period of the test samples. Stability is assessed by comparing the stability samples with their theoretical concentration. The stability samples are the mAb 26.4 QC1 and QC3 in vehicle that have been stored in a freezer set to maintain −80° C. for the appropriate time duration. Samples designated for use as long-term stability assessment samples are shown to meet acceptance criteria (aiming for ±10% mean relative error) prior to storage. Long-term stability samples are analyzed using a freshly prepared standard curve and assay acceptance QC samples. Stability is considered acceptable if the mean concentration of the stability samples is within ±20% RE (%) and ≤20% CV (%) at each concentration.

### Nucleotide and Amino Acid Sequences

The nucleotide (DNA) and amino acid sequences of the present disclosure are provided in Table 19 below.

TABLE 19
Nucleotide and amino acid sequences.

| SEQ ID NO | Nucleotide/Amino Acid Sequence |
|---|---|
| SEQ ID | GPNICTTRGVSSCQQCLAVSPMCAWCSDEALPLGSPRCDLKENLLKDNCA |
| NO: 1 | PESIEFPVSEARVLEDRPLSDKGSGDSSQVTQVSPQRIALRLRPDDSKNF |
| | SIQVRQVEDYPVDIYYLMDLSYSMKDDLWSIQNLGTKLATQMRKLTSNLR |
| | IGFGAFVDKPVSPYMYISPPEALENPCYDMKTTCLPMFGYKHVLTLTDQV |
| | TRENEEVKKQSVSRNRDAPEGGFDAIMQATVCDEKIGWRNDASHLLVETT |
| | DAKTHIALDGRLAGIVQPNDGQCHVGSDNHYSASTTMDYPSLGLMTEKLS |
| | QKNINLIFAVTENVVNLYQNYSELIPGTTVGVLSMDSSNVLQLIVDAYGK |
| | IRSKVELEVRDLPEELSLSFNATCLNNEVIPGLKSCMGLKIGDTVSFSIE |
| | AKVRGCPQEKEKSFTIKPVGFKDSLIVQVTFDCDCACQAQAEPNSHRCNN |
| | GNGTFECGVCRCGPGWLGSQCECSEEDYRPSQQDECSPREGQPVCSQRGE |
| | CLCGQCVCHSSDFGKITGKYCECDDFSCVRYKGEMCSGHGQCSCGDCLCD |
| | SDWTGYYCNCTTRTDTCMSSNGLLCSGRGKCECGSCVCIQPGSYGDTCEK |
| | CPTCPDACTFKKECVECKKEDREPYMTENTCNRYCRDEIESVKELKDTGK |
| | DAVNCTYKNEDDCVVRFQYYEDSSGKSILYVVEEPECPKGPDILVVLLSV |
| | MGAILLIGLAALLIWKLLITIHDRKEFAKFEEERARAKWDTANNPLYKEA |
| | TSTFTNITYRGT |
| SEQ ID | GPNICTTRGVSSCQQCLAVSPMCAWCSDEALPLGSPRCDLKENLLKDNCA |
| NO: 2 | PESIEFPVSEARVLEDRPLSDKGSGDSSQVTQVSPQRIALRLRPDDSKNF |
| | SIQVRQVEDGNVDLVFLFDGSMSLQPDEFQKILDFMKDVMKKLSNTSYQF |
| | AAVQFSTSYKTEFDFSDYVKWKDPDALLKHVKHMLLLTNTFGAINYVATE |
| | VFREELGARPDATKVLIIITDGEATDSGNIDAAKDIIRYIIGIGKHFQTK |
| | ESQETLHKFASKPASEFVKILDTFEKLKDLFTELQKKIRSKVELEVRDLP |
| | EELSLSFNATCLNNEVIPGLKSCMGLKIGDTVSFSIEAKVRGCPQEKEKS |
| | FTIKPVGFKDSLIVQVTFDCDCACQAQAEPNSHRCNNGNGTFECGVCRCG |
| | PGWLGSQCECSEEDYRPSQQDECSPREGQPVCSQRGECLCGQCVCHSSDE |
| | GKITGKYCECDDFSCVRYKGEMCSGHGQCSCGDCLCDSDWTGYYCNCTTR |
| | TDTCMSSNGLLCSGRGKCECGSCVCIQPGSYGDTCEKCPTCPDACTFKKE |
| | CVECKKFDRGALHDENTCNRYCRDEIESVKELKDTGKDAVNCTYKNEDDC |
| | VVRFQYYEDSSGKSILYVVEEPECP |
| SEQ ID | MRARPRPRPLWVTVLALGALAGVGVG |
| NO: 3 | |
| SEQ ID | QVQLQQSGPGLVKPSQTLSLTCAISGDSVSSNSAAWNWIRQSPSRGLEWL |
| NO: 4 | GRTYFRSNWYNDYAASVKSRITINQDTSKNQLSLQLNSVTPEDTAMYYCA |
| | RDGAWGGSSWWPGLPHHYYSGMDVWGQGTTVTVSS |
| SEQ ID | EIVLTQSPATLSLSPGERATLSCRASQSVSSYLAWYQQKPGQAPRLLIYD |
| NO: 5 | ASKRATGIPARFSGSGSGTDFSLTIRSLEPEDFAVYYCQQRSDWQGLTFG |
| | GGTKVEIK |
| SEQ ID | GDSVSSNSAA |
| NO: 6 | |
| SEQ ID | TYFRSNWYN |
| NO: 7 | |
| SEQ ID | ARDGAWGGSSWWPGLPHHYYSGMDV |
| NO: 8 | |
| SEQ ID | QSVSSY |
| NO: 9 | |
| SEQ ID | DAS |
| NO: 10 | |
| SEQ ID | QQRSDWQGLT |
| NO: 11 | |
| SEQ ID | ATGAGAGCCCGCCCCAGACCTAGACCACTTTGGGCCACCGTCCTTGCCCT |
| NO: 12 | CGGAGCCCTTGCCGGAGTGGGAGTGGGCGGACCAAACATCTGTACTACCC |
| | GGGGCGTCAGCTCCTGTCAGCAGTGCCTGGCTGTGTCTCCTATGTGCGCT |
| | TGGTGCTCCGACGAAGCGCTGCCCCTCGGCTCACCCCGCTGCGACTTGAA |
| | GGAGAACCTGTTGAAGGACAACTGCGCACCCGAATCCATCGAGTTCCCGG |
| | TGTCCGAAGCCCGGGTCTTGGAAGATCGGCCGCTGTCCGACAAAGGGTCC |
| | GGGGACAGCTCACAGGTCACCCAAGTGTCGCCTCAACGGATCGCACTGCG |
| | GCTCCGCCCGGACGACAGCAAGAACTTCTCAATCCAAGTCCGCCAAGTGG |
| | AGGACGGGAATGTGGATCTCGTGTTCCTGTTCGACGGCTCCATGTCACTC |
| | CAGCCTGACGAATTCCAGAAGATTCTAGACTTCATGAAGGACGTGATGAA |
| | GAAGCTGTCGAACACCTCCTACCAATTCGCGGCCGTGCAGTTCAGCACCT |
| | CCTACAAGACTGAGTTTGACTTCTCCGACTACGTGAAGTGGAAGGACCCG |
| | GATGCCCTGCTGAAGCACGTGAAGCACATGCTTCTGCTCACTAACACGTT |
| | TGGCGCGATCAACTACGTGGCGACTGAAGTGTTTAGAGAAGAGTTGGGAG |
| | CCAGGCCTGATGCCACCAAAGTGCTGATAATCATCACTGACGGAGAGGCC |
| | ACTGACAGCGGAAACATCGACGCCGCAAAGGACATCATTCGGTACATCAT |
| | TGGCATCGGAAAGCACTTCCAGACCAAAGAATCCCAGGAAACCCTGCATA |
| | AGTTCGCCTCGAAACCGGCGTCGGAGTTCGTGAAGATCCTCGATACCTTT |
| | GAAAAGCTGAAGGACCTGTTCACCGAGCTCCAGAAGAAGATTCGGTCCAA |
| | GGTCGAACTGGAGGTCCGCGACCTCCCCGAGGAGCTCTCCCTGAGTTTCA |
| | ACGCTACCTGTCTTAACAATGAGGTCATCCCTGGACTGAAGTCCTGCATG |
| | GGTCTGAAGATCGGCGATACCGTGTCATTCTCTATCGAAGCCAAAGTCCG |
| | GGGTTGTCCGCAAGAAAAGGAGAAGTCCTTCACCATCAAGCCCGTGGGAT |
| | TCAAGGATAGCCTGATCGTTCAAGTCACTTTCGACTGCGACTGCGCCTGC |
| | CAAGCGCAGGCCGAGCCCAACTCGCATAGATGCAACAACGGCAACGGAAC |
| | TTTCGAATGTGGCGTCTGTAGATGTGGCCCGGGCTGGCTGGGAAGCCAGT |
| | GCGAATGCTCAGAGGAGGACTACCGCCCGAGCCAGCAGGATGAGTGCAGC |
| | CCCCGCGAGGGACAGCCCGTGTGCAGCCAGCGGGGCGAATGTCTGTGTGG |
| | TCAATGCGTGTGCCACTCCTCCGATTTCGGAAAGATTACCGGAAAGTACT |
| | GCGAATGCGACGACTTCTCCTGCGTGCGGTACAAGGGGGAAATGTGTTCC |
| | GGTCATGGGCAGTGCTCCTGCGGCGACTGCCTGTGCGATAGCGACTGGAC |
| | CGGCTACTATTGCAATTGCACCACGAGGACCGACACTTGCATGTCCTCCA |
| | ACGGACTGCTGTGCTCCGGCCGGGGAAAATGCGAATGTGGCTCGTGCGTG |
| | TGCATTCAGCCCGGATCCTACGGGGATACTTGCGAAAAGTGCCCGACATG |
| | CCCGGACGCGTGTACCTTCAAGAAGGAATGCGTGGAGTGCAAGAAATTTG |
| | ACCGGGGGGCCTTGCACGACGAGAACACATGCAACCGCTACTGTCGCGAC |
| | GAAATCGAGTCGGTGAAAGAACTGAAGGACACGGGGAAAGACGCTGTGAA |
| | CTGCACCTACAAGAACGAAGATGACTGTGTCGTGCGGTTTCAGTACTATG |
| | AAGATTCGTCCGGAAAGTCAATTCTCTACGTGGTGGAAGAACCCGAATGC |
| | CCGGGCGGTGGACTGGATGTGCTGTTCCAAGGGCCAGTGGAAGATCAGGT |
| | CGACCCTAGGCTGATCGACGGAAAGGGATCGGGTCACCACCATCACCACC |
| | ATGGAACCACCGGTTGGCGCGGGGGACACGTGGTGGAGGGTCTGGCTGGA |
| | GAGCTGGAACAGCTGAGGGCCCGCCTGGAGCACCACCCGCAAGGACAGCG |
| | GGAGCCAGCATGATAA |
| SEQ ID | MRARPRPRPLWATVLALGALAGVGVGGPNICTTRGVSSCQQCLAVSPMCA |
| NO: 13 | WCSDEALPLGSPRCDLKENLLKDNCAPESIEFPVSEARVLEDRPLSDKGS |
| | GDSSQVTQVSPQRIALRLRPDDSKNFSIQVRQVEDGNVDLVELEDGSMSL |
| | QPDEFQKILDFMKDVMKKLSNTSYQFAAVQFSTSYKTEFDFSDYVKWKDP |
| | DALLKHVKHMLLLTNTFGAINYVATEVFREELGARPDATKVLIIITDGEA |
| | TDSGNIDAAKDIIRYIIGIGKHFQTKESQETLHKFASKPASEFVKILDTF |
| | EKLKDLFTELQKKIRSKVELEVRDLPEELSLSFNATCLNNEVIPGLKSCM |
| | GLKIGDTVSFSIEAKVRGCPQEKEKSFTIKPVGFKDSLIVQVTFDCDCAC |
| | QAQAEPNSHRCNNGNGTFECGVCRCGPGWLGSQCECSEEDYRPSQQDECS |
| | PREGQPVCSQRGECLCGQCVCHSSDFGKITGKYCECDDFSCVRYKGEMCS |
| | GHGQCSCGDCLCDSDWTGYYCNCTTRTDTCMSSNGLLCSGRGKCECGSCV |
| | CIQPGSYGDTCEKCPTCPDACTFKKECVECKKFDRGALHDENTCNRYCRD |
| | EIESVKELKDTGKDAVNCTYKNEDDCVVRFQYYEDSSGKSILYVVEEPEC |
| | PGGGLDVLFQGPVEDQVDPRLIDGKGSGHHHHHHGTTGWRGGHVVEGLAG |
| | ELEQLRARLEHHPQGQREPA |
| SEQ ID | GPNICTTRGVSSCQQCLAVSPMCAWCSDEALPLGSPRCDLKENLLKDNCA |
| NO: 14 | PESIEFPVSEARVLEDRPLSDKGSGDSSQVTQVSPQRIALRLRPDDSKNF |
| | SIQVRQVEDGNVDLVFLFDGSMSLQPDEFQKILDEMKDVMKKLSNTSYQF |
| | AAVQFSTSYKTEFDFSDYVKWKDPDALLKHVKHMLLLTNTFGAINYVATE |
| | VFREELGARPDATKVLIIITDGEATDSGNIDAAKDIIRYIIGIGKHFQTK |
| | ESQETLHKFASKPASEFVKILDTFEKLKDLFTELQKKIRSKVELEVRDLP |
| | EELSLSFNATCLNNEVIPGLKSCMGLKIGDTVSFSIEAKVRGCPQEKEKS |
| | FTIKPVGFKDSLIVQVTFDCDCACQAQAEPNSHRCNNGNGTFECGVCRCG |
| | PGWLGSQCECSEEDYRPSQQDECSPREGQPVCSQRGECLCGQCVCHSSDF |
| | GKITGKYCECDDFSCVRYKGEMCSGHGQCSCGDCLCDSDWTGYYCNCTTR |
| | TDTCMSSNGLLCSGRGKCECGSCVCIQPGSYGDTCEKCPTCPDACTEKKE |
| | CVECKKFDRGALHDENTCNRYCRDEIESVKELKDTGKDAVNCTYKNEDDC |
| | VVRFQYYEDSSGKSILYVVEEPECPGGGLDVLFQGPVEDQVDPRLIDGKG |
| | SGHHHHHHGTTGWRGGHVVEGLAGELEQLRARLEHHPQGQREPA |

### REFERENCES

Abhinandan K R and Martin A C R. Analysis and improvements to Kabat and structurally correct numbering of antibody variable domains. Mol. Immunol. 45:3832-9 (2008).
Almagro J C. Identification of differences in the specificity-determining residues of antibodies that recognize antigens of different size: implications for the rational design of antibody repertoires. J. Mol. Recognit. 17:132-43 (2004).
Campbell K, et al. A modified rapid monoclonal antibody-specific immobilization of platelet antigen assay for the detection of human platelet antigen (HPA) antibodies: a multicentre evaluation. Vox Sanguinis 93:289-97 (2007).
Chothia C. and Lesk M. Canonical structures for the hypervariable regions of immunoglobulins. J. Mol. Biol. 196:901-17 (1987).
Eksteen M, et al. Characterization of a human platelet antigen-1a-specific monoclonal antibody derived from a B cell from a woman alloimmunized in pregnancy. J. Immunol. 194:5751-60 (2015).
Ghevaert C, et al. Management and outcome of 200 cases of fetomaternal alloimmune thrombocytopenia. Transfusion 47:901-10 (2007).
Kiefel V, et al. Monoclonal antibody-specific immobilization of platelet antigens (MAIPA): a new tool for the identification of platelet-reactive antibodies. Blood 70:1722-26 (1987).
Lee J-O, et al. Two conformations of the integrin A-domain (I-domain): a pathway for activation? Structure 3:1333-40 (1995).
Lefranc M P, et al. IMGT unique numbering for immunoglobulin and T cell receptor variable domains and Ig superfamily V-like domains. Dev. Comp. Immunol. 27:55-77 (2003).
Martin A C R. Antibody Engineering. Kontermann R and Dubel S eds., Springer-Verlag, Berlin, 2:33-51 (2010).
Newman P J, et al. The human platelet alloantigens, Pl^{A1} and Pl^{A2}, are associated with a leucine³³/proline³³ amino acid polymorphism in membrane glycoprotein IIIa, and are distinguishable by DNA typing. J. Clin. Invest. 83:1778-81 (1989).
Takada Y, et al. The integrins. Genome Biol. 8:215.1-215.9 (2007).
Takagi T, et al. The Role of the Specificity-Determining Loop of the Integrin β Subunit I-like Domain in Autonomous Expression, Association with the α Subunit, and Ligand Binding. Biochem. 41:4339-47 (2002).
Thinn A M M, et al. Autonomous conformational regulation of β₃ integrin and the conformation-dependent property of HPA-1a alloantibodies. Proc. Nat'l. Acad. Sci. U.S.A. 115:E9105-14; Suppl. Materials 1-7 (2018).
WHO International Standard: Anti-HPA-1a Standard (100 IU). NIBSC code: 03/152. National Institute for Biological Standards and Control, Hertfordshire, United Kingdom.
Wu T T and Kabat E. An analysis of the sequences of the variable regions of Bence Jones proteins and myeloma light chains and their implications for antibody complementarity. J. Erp. Med. 132:211-50 (1970).
Zhou D, et al. Structure of an extended β₃ integrin. Blood 132:962-72 (2018).

The present invention is further described by the following claims.

1. A method of quantifying anti-HPA-1a antibodies in a sample, the method comprising:
(a) contacting the sample with a capture reagent that binds to the anti-HPA-1a antibodies to form a detection complex, wherein the capture reagent comprises a modified integrin β₃; and
(b) measuring an amount of the detection complex, wherein the amount of the detection complex indicates the quantity of the anti-HPA-1a antibodies in the sample.

2. A method of determining potency of anti-HPA-1a antibodies in a sample, the method comprising:
(a) contacting the sample with a capture reagent that binds to the anti-HPA-1a antibodies to form a detection complex, wherein the capture reagent comprises a modified integrin β₃; and
(b) measuring an amount of the detection complex, wherein the amount of the detection complex indicates the potency of the anti-HPA-1a antibodies in the sample.

3. A method of measuring pharmacokinetics of anti-HPA-1a antibodies administered to a subject, the method comprising:
(a) contacting a sample from the subject with a capture reagent that binds to the anti-HPA-1a antibodies to form a detection complex, wherein the capture reagent comprises a modified integrin β₃; and
(b) measuring an amount of the detection complex, wherein the amount of the detection complex indicates the quantity of the anti-HPA-1a antibodies in the sample;
wherein the quantity of the anti-HPA-1a antibodies in the sample is used to calculate the pharmacokinetics of the anti-HPA-1a antibodies administered to the subject.

4. The method of any one of claims 1-3, wherein the modified integrin β₃ is prepared by replacing the I-like domain of integrin β₃ (βI) with the I domain of an integrin α (αI).

5. The method of any one of claims 1-4, wherein the modified integrin β₃ comprises the amino acid sequence of SEQ ID NO: 2.

6. The method of any one of claims 1-5, wherein the modified integrin β₃ consists of the amino acid sequence of SEQ ID NO: 2.

7. The method of any one of claims 1-6, wherein the modified integrin β₃ is deglycosylated.

8. The method of any one of claims 1-7, wherein the capture reagent comprises a detectable label attached to the modified integrin β₃, and measuring the amount of the detection complex comprises measuring the amount of the detectable label.

9. The method of any one of claims 1-7, further comprising contacting the sample with a detecting reagent that binds to the anti-HPA-1a antibodies to become part of the detection complex.

10. The method of claim 9, wherein the detecting reagent comprises an antibody or antigen-binding fragment thereof that binds to the anti-HPA-1a antibodies.

11. The method of claim 10, wherein the detecting reagent comprises a detectable label attached to the antibody or antigen-binding fragment thereof, and measuring the amount of the detection complex comprises measuring the amount of the detectable label.

12. The method of claim 8 or 11, wherein the detectable label is selected from a group consisting of an enzyme, a fluorescent label, a chemiluminescent label, a bioluminescent label, a radioactive label, or a combination thereof.

13. The method of any one of claims 8, 11, or 12, wherein the detectable label comprises an enzyme.

14. The method of claim 12 or 13, wherein the detectable label comprises horseradish peroxidase.

15. The method of claim 8 or 11, wherein the amount of the detectable label is measured using light scattering, optical absorbance, fluorescence, chemiluminescence, electrochemiluminescence, or radioactivity.

16. The method of any one of claims 1-15, wherein the capture reagent is immobilized on a solid support.

17. The method of claim 16, wherein the solid support comprises a surface, particles, or a porous matrix.

18. The method of claim 17, wherein the solid support comprises a multi-well plate.

19. The method of any one of claims 1-3, further comprising attaching a first fragment of a reporter protein to the anti-HPA-1a antibodies.

20. The method of claim 19, wherein the capture reagent comprises a second fragment of the reporter protein attached to the modified integrin β₃, and measuring the amount of the detection complex comprises measuring the amount of the reporter proteins that are formed when the capturing reagents bind to the anti-HPA-1a antibodies.

21. The method of claim 19, further comprising contacting the sample with a detecting reagent that binds to the anti-HPA-1a antibodies to become part of the detection complex.

22. The method of claim 21, wherein the detecting reagent comprises an antibody or antigen-binding fragment thereof that binds to the anti-HPA-1a antibodies, and a second fragment of the reporter protein attached to the antibody or antigen-binding fragment thereof; and wherein measuring the amount of the detection complex comprises measuring the amount of the reporter proteins that are formed when the detecting reagents bind to the anti-HPA-1a antibodies.

23. The method of any one of claims 19-22, wherein the reporter protein is selected from beta-lactamase, dihydrofolate reductase, focal adhesion kinase, Gal4, green fluorescent protein, horseradish peroxidase, infrared fluorescent protein IFP1.4, lacZ (beta-galactosidase), luciferase, tobacco etch virus protease, and ubiquitin.

24. The method of claim 20 or 22, wherein the amount of the reporter protein is measured using light scattering, optical absorbance, fluorescence, chemiluminescence, electrochemiluminescence, or radioactivity.

25. The method of any one of claims 1-3, wherein the capture reagent comprises an oligonucleotide attached to the modified integrin β₃, and measuring the amount of the detection complex comprises measuring the amount of the oligonucleotide.

26. The method of claim 25, further comprising contacting the sample with a detecting reagent that binds to the anti-HPA-1a antibodies to become part of the detection complex.

27. The method of claim 26, wherein the detecting reagent comprises an antibody or antigen-binding fragment thereof that binds to the anti-HPA-1a antibodies, and an oligonucleotide attached to the antibody or antigen-binding fragment thereof; and wherein measuring the amount of the detection complex comprises measuring the amount of the oligonucleotide.

28. The method of claim 25 or 27, wherein the amount of the oligonucleotide is measured using real-time polymerase chain reaction.

29. The method of any one of claims 10-18, 22-24, 27, or 28, wherein the antibody or antigen-binding fragment thereof comprises an anti-immunoglobulin G (IgG) antibody, anti-IgM antibody, anti-IgD antibody, anti-IgE antibody, or anti-IgA antibody.

30. The method of any one of claims 10-18, 22-24, or 27-29, wherein the antibody or antigen-binding fragment thereof comprises an anti-IgG antibody.

31. The method of any one of claims 1-30, wherein the sample comprises a biological fluid selected from whole blood, serum, and plasma.

32. The method of any one of claims 1-31, wherein the anti-HPA-1a antibodies are polyclonal antibodies.

33. The method of any one of claims 1-31, wherein the anti-HPA-1a antibodies are monoclonal antibodies.

34. The method according to claim 33, wherein the monoclonal antibodies comprise (i) a variable heavy (VH) complementarity determining region (CDR) 1 that has the amino acid sequence of SEQ ID NO:6, (ii) a VH CDR2 that has the amino acid sequence of SEQ ID NO:7, and (iii) a VH CDR3 that has the amino acid sequence of SEQ ID NO:8, (iv) a variable light (VL) CDR1 that has the amino acid sequence of SEQ ID NO:9, (v) a VL CDR2 that has the amino acid sequence of SEQ ID NO:10, and (vi) a VL CDR3 that has the amino acid sequence of SEQ ID NO:11.

35. The method according to claim 34, wherein the monoclonal antibodies comprise a heavy chain variable region comprising SEQ ID NO:4, or a sequence having at least 80% sequence identity thereto; and a light chain variable region comprising SEQ ID NO:5, or a sequence having at least 80% sequence identity thereto.

36. The method of claim 33, wherein the monoclonal antibodies are mAb 26.4.

37. A method of quantifying anti-HPA-1a antibodies in a sample, the method comprising:
(a) contacting the sample with a capture reagent that binds to the anti-HPA-1a antibodies, wherein the capture reagent comprises a modified integrin β₃ comprising the amino acid sequence of SEQ ID NO: 2;
(b) contacting the sample with a detecting reagent that binds to the anti-HPA-1a antibodies, wherein the detecting reagent comprises an antibody or antigen-binding fragment thereof that binds to the anti-HPA-1a antibodies and a detectable label attached thereto, and wherein the anti-HPA-1a antibodies, the capture reagent, and the detecting reagent forms a detection complex; and
(c) measuring an amount of the detection complex, wherein the amount of the detection complex indicates the quantity of the anti-HPA-1a antibodies in the sample.

38. A method of determining potency of anti-HPA-1a antibodies in a sample, the method comprising:
(a) contacting the sample with a capture reagent that binds to the anti-HPA-1a antibodies, wherein the capture reagent comprises a modified integrin β₃ comprising the amino acid sequence of SEQ ID NO: 2;
(b) contacting the sample with a detecting reagent that binds to the anti-HPA-1a antibodies, wherein the detecting reagent comprises an antibody or antigen-binding fragment thereof that binds to the anti-HPA-1a antibodies and a detectable label attached thereto, and wherein the anti-HPA-1a antibodies, the capture reagent, and the detecting reagent forms a detection complex; and
(c) measuring an amount of the detection complex, wherein the amount of the detection complex indicates the potency of the anti-HPA-1a antibodies in the sample.

39. A method of measuring pharmacokinetics of anti-HPA-1a antibodies administered to a subject, the method comprising:
(a) contacting a sample from the subject with a capture reagent that binds to the anti-HPA-1a antibodies, wherein the capture reagent comprises a modified integrin β₃ comprising the amino acid sequence of SEQ ID NO: 2;
(b) contacting the sample with a detecting reagent that binds to the anti-HPA-1a antibodies, wherein the detecting reagent comprises an antibody or antigen-binding fragment thereof that binds to the anti-HPA-1a antibodies and a detectable label attached thereto, and wherein the anti-HPA-1a antibodies, the capture reagent, and the detecting reagent forms a detection complex; and
(c) measuring an amount of the detection complex, wherein the amount of the detection complex indicates the quantity of the anti-HPA-1a antibodies in the sample.

40. A kit for an assay for quantifying anti-HPA-1a antibodies in a sample, the kit comprising:
(i) a capture reagent comprising a modified integrin β₃; and
(ii) instructions on how to perform the assay using the capture reagent.

41. The kit of claim 40, wherein the modified integrin β₃ is prepared by replacing the I-like domain of integrin β₃ (βI) with the I domain of an integrin α (αI).

42. The kit of claim 40 or 41, wherein the modified integrin β₃ comprises the amino acid sequence of SEQ ID NO: 2.

43. The kit of any one of claims 40-42, wherein the capture reagent further comprises a detectable label attached to the modified integrin cβ₃.

44. The kit of any one of claims 40-42, further comprising a detecting reagent comprising an antibody or antigen-binding fragment thereof that binds to anti-HPA-1a antibodies.

45. The kit of claim 44, wherein the detecting reagent further comprises a detectable label attached to the antibody or antigen-binding fragment thereof.

46. The kit of claim 43 or 45, wherein the detectable label comprises an enzyme.

47. The kit of claim 46, further comprising a substrate required by the enzyme to produce a detectable signal.

48. The kit of any one of claims 40-47, wherein the capture reagent is immobilized on a solid support.

49. The kit of any one of claims 40-47, further comprising a solid support on which to immobilize the capture reagent.

50. The kit of any one of claims 40-49, further comprising washing buffers, incubation buffers, blocking agents, or a combination thereof.

51. A method of manufacturing a pharmaceutical composition comprising an anti-HPA-1a antibody, the method comprising:
(a) admixing an anti-HPA-1a antibody and a pharmaceutically acceptable carrier to prepare a composition;
(b) measuring potency of the anti-HPA-1a antibody in the composition according to the method of claim 2 to determine the concentration of anti-HPA-1a antibody in the composition; and
(c) optionally adjusting the concentration of anti-HPA-1a antibody in the composition to a specification concentration for a pharmaceutical composition comprising anti-HPA-1a antibody.

52. The method according to claim 51, wherein the pharmaceutical composition comprises a monoclonal anti-HPA-1a antibody comprising (i) a variable heavy (VH) complementarity determining region (CDR) 1 that has the amino acid sequence of SEQ ID NO:6, (ii) a VH CDR2 that has the amino acid sequence of SEQ ID NO:7, and (iii) a VH CDR3 that has the amino acid sequence of SEQ ID NO:8, (iv) a variable light (VL) CDR1 that has the amino acid sequence of SEQ ID NO:9, (v) a VL CDR2 that has the amino acid sequence of SEQ ID NO:10, and (vi) a VL CDR3 that has the amino acid sequence of SEQ ID NO:11.

53. The method according to claim 52, wherein the monoclonal anti-HPA-1a antibody comprises a heavy chain variable region comprising SEQ ID NO:4, or a sequence having at least 80% sequence identity thereto; and wherein the monoclonal anti-HPA-1a antibody comprises a light chain variable region comprising SEQ ID NO:5, or a sequence having at least 80% sequence identity thereto.

54. The method of claim 51, wherein the pharmaceutical composition comprises a polyclonal anti-HPA-1a antibody.

55. The method of claim 54, wherein the polyclonal anti-HPA-1a antibody is from plasma of one or more HPA-1a-negative subjects immunized with HPA-1a.

56. The method of claim 55, wherein the subject was alloimmunized with HPA-1a.

57. The method of claim 55, wherein the subject was immunized with HPA-1a positive platelets or with a purified or recombinant preparation HPA-1a antigen.

58. The method of any one of claims 55-57, further comprising clearing the polyclonal anti-HPA-1a antibody of viruses.


SEQUENCE LISTING (14 sequences)

SEQ ID NO: 1 - protein, 762 aa, synthetic construct
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDREPYMTENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 2 - protein, 625 aa, synthetic construct
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECP

SEQ ID NO: 3 - protein, 26 aa, synthetic construct
MRARPRPRPLWVTVLALGALAGVGVG

SEQ ID NO: 4 - protein, 135 aa, synthetic construct
QVQLQQSGPGLVKPSQTLSLTCAISGDSVSSNSAAWNWIRQSPSRGLEWLGRTYFRSNWYNDYAASVKSRITINQDTSKNQLSLQLNSVTPEDTAMYYCARDGAWGGSSWWPGLPHHYYSGMDVWGQGTTVTVSS

SEQ ID NO: 5 - protein, 108 aa, synthetic construct
EIVLTQSPATLSLSPGERATLSCRASQSVSSYLAWYQQKPGQAPRLLIYDASKRATGIPARFSGSGSGTDFSLTIRSLEPEDFAVYYCQQRSDWQGLTFGGGTKVEIK

SEQ ID NO: 6 - protein, 10 aa, synthetic construct
GDSVSSNSAA

SEQ ID NO: 7 - protein, 9 aa, synthetic construct
TYFRSNWYN

SEQ ID NO: 8 - protein, 25 aa, synthetic construct
ARDGAWGGSSWWPGLPHHYYSGMDV

SEQ ID NO: 9 - protein, 6 aa, synthetic construct
QSVSSY

SEQ ID NO: 10 - skipped

SEQ ID NO: 11 - protein, 10 aa, synthetic construct
QQRSDWQGLT

SEQ ID NO: 12 - DNA, 2166 bp, synthetic construct
  source 1..2166: mol_type = other DNA
atgagagcccgccccagacctagaccactttgggccaccgtccttgccctcggagcccttgccggagtgggagtgggcggaccaaacatctgtactacccggggcgtcagctcctgtcagcagtgcctggctgtgtctcctatgtgcgcttggtgctccgacgaagcgctgcccctcggctcaccccgctgcgacttgaaggagaacctgttgaaggacaactgcgcacccgaatccatcgagttcccggtgtccgaagcccgggtcttggaagatcggccgctgtccgacaaagggtccggggacagctcacaggtcacccaagtgtcgcctcaacggatcgcactgcggctccgcccggacgacagcaagaacttctcaatccaagtccgccaagtggaggacgggaatgtggatctcgtgttcctgttcgacggctccatgtcactccagcctgacgaattccagaagattctagacttcatgaaggacgtgatgaagaagctgtcgaacacctcctaccaattcgcggccgtgcagttcagcacctcctacaagactgagtttgacttctccgactacgtgaagtggaaggacccggatgccctgctgaagcacgtgaagcacatgcttctgctcactaacacgtttggcgcgatcaactacgtggcgactgaagtgtttagagaagagttgggagccaggcctgatgccaccaaagtgctgataatcatcactgacggagaggccactgacagcggaaacatcgacgccgcaaaggacatcattcggtacatcattggcatcggaaagcacttccagaccaaagaatcccaggaaaccctgcataagttcgcctcgaaaccggcgtcggagttcgtgaagatcctcgatacctttgaaaagctgaaggacctgttcaccgagctccagaagaagattcggtccaaggtcgaactggaggtccgcgacctccccgaggagctctccctgagtttcaacgctacctgtcttaacaatgaggtcatccctggactgaagtcctgcatgggtctgaagatcggcgataccgtgtcattctctatcgaagccaaagtccggggttgtccgcaagaaaaggagaagtccttcaccatcaagcccgtgggattcaaggatagcctgatcgttcaagtcactttcgactgcgactgcgcctgccaagcgcaggccgagcccaactcgcatagatgcaacaacggcaacggaactttcgaatgtggcgtctgtagatgtggcccgggctggctgggaagccagtgcgaatgctcagaggaggactaccgcccgagccagcaggatgagtgcagcccccgcgagggacagcccgtgtgcagccagcggggcgaatgtctgtgtggtcaatgcgtgtgccactcctccgatttcggaaagattaccggaaagtactgcgaatgcgacgacttctcctgcgtgcggtacaagggggaaatgtgttccggtcatgggcagtgctcctgcggcgactgcctgtgcgatagcgactggaccggctactattgcaattgcaccacgaggaccgacacttgcatgtcctccaacggactgctgtgctccggccggggaaaatgcgaatgtggctcgtgcgtgtgcattcagcccggatcctacggggatacttgcgaaaagtgcccgacatgcccggacgcgtgtaccttcaagaaggaatgcgtggagtgcaagaaatttgaccggggggccttgcacgacgagaacacatgcaaccgctactgtcgcgacgaaatcgagtcggtgaaagaactgaaggacacggggaaagacgctgtgaactgcacctacaagaacgaagatgactgtgtcgtgcggtttcagtactatgaagattcgtccggaaagtcaattctctacgtggtggaagaacccgaatgcccgggcggtggactggatgtgctgttccaagggccagtggaagatcaggtcgaccctaggctgatcgacggaaagggatcgggtcaccaccatcaccaccatggaaccaccggttggcgcgggggacacgtggtggagggtctggctggagagctggaacagctgagggcccgcctggagcaccacccgcaaggacagcgggagccagcatgataa

SEQ ID NO: 13 - protein, 720 aa, synthetic construct
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPGGGLDVLFQGPVEDQVDPRLIDGKGSGHHHHHHGTTGWRGGHVVEGLAGELEQLRARLEHHPQGQREPA

SEQ ID NO: 14 - protein, 694 aa, synthetic construct
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPGGGLDVLFQGPVEDQVDPRLIDGKGSGHHHHHHGTTGWRGGHVVEGLAGELEQLRARLEHHPQGQREPA